(19) United States
(12) Patent Application Publication
(10) Pub. No.: US 2026/0130903 A1
(43) Pub. Date: May 14, 2026

(54) METHODS OF ADMINISTERING A VOLTAGE-GATED POTASSIUM CHANNEL OPENER

(71) Applicant: Xenon Pharmaceuticals Inc., Burnaby (CA)

(72) Inventors: Gregory N. Beatch, Burnaby (CA); Christopher Christopher Kenney, Duxbury, MA (US); Joseph William McIntosh, Bridgewater, NJ (US); Rostam Namdari, Burnaby (CA); Aleksandrs Odinecs, Burnaby (CA)

(73) Assignee: Xenon Pharmaceuticals Inc., Burnaby (CA)

(21) Appl. No.: 19/412,760

(22) Filed: Dec. 8, 2025

Related U.S. Application Data
(63) Continuation of application No. PCT/US2025/054685, filed on Nov. 7, 2025.
(60) Provisional application No. 63/717,948, filed on Nov. 8, 2024.

(51) Int. Cl. A61K 31/472; A61K 45/06

(52) U.S. Cl. CPC A61K 31/472; A61K 45/06

(57) ABSTRACT

The present disclosure provides methods related to the co-administration of a cytochrome P450 3A4 (CYP3A4) inhibitor and Compound A (azetukalner). The combination of the CYP3A4 inhibitor and azetukalner is useful in the treatment and/or prevention of diseases, disorders, and conditions (e.g., diseases, disorders, and conditions associated with Kv7 potassium channel dysfunction).

### RELATED APPLICATIONS

This application claims priority under 35 U.S.C. § 119(e) to U.S. Provisional Patent Applications, U.S. Ser. No. 63/717,948, filed Nov. 8, 2024, which is incorporated herein by reference in its entirety.

### BACKGROUND

Voltage-gated potassium channels, including the voltage-gated potassium channels (e.g., Kv7.2 and Kv7.3 (Kv7.2/Kv7.3)), are important in controlling neuronal excitability. Kv7.2/Kv7.3 underlie the neuronal “M-current,” named according to its initial characterization as a neuronal current decreased in response to muscarinic/cholinergic agonists (see Brown, D. A. et al., Nature (1980), 283:673-676). The M-current is a non-inactivating, hyperpolarizing current known to act as a brake on neuronal hyperexcitability. Consequently, a decrease in the Kv7.2-mediated M-current, for example through genetic loss-of-function, can cause neuronal depolarization and an increase in membrane and neuronal excitability that can lead to action potential bursts that manifest as, e.g., epileptic seizures. In contrast, an increase in the Kv7.2-mediated M-current can hyperpolarize the cell membrane and thereby reduce neuronal excitability and prevent the initiation and propagation of action potential bursts and the resultant seizures. Enhancing the open state of potassium (e.g., Kv7.2/Kv7.3) channels in neurons favors a hyperpolarized resting state, which reduces rapid action potential spiking (i.e., burst firing). Such enhancement can provide a stabilizing effect on excitable, particularly hyper-excitable, neurons and can therefore be useful in treating certain seizure disorders.

While significant advances have been made in this field, there remains a substantial need for improved methods for potentiating voltage-gated potassium channels, including the voltage-gated potassium channels (e.g., Kv7.2/Kv7.3). Such compounds and methods can be used to treat diseases, disorders, and conditions in which potassium channel dysfunction is implicated.

The cytochrome P450 enzyme system (CYP450) plays a predominant role in the metabolism of drugs, such as azetukalner, often transforming active substances into inactive metabolites that can be excreted from the body. In certain cases, the metabolism of certain drugs by CYP450 enzymes (e.g., Cytochrome P450 3A4 (CYP3A4)) can alter their pharmacokinetic profile and result in sub-therapeutic plasma levels of those drugs over time. On the other hand, a CYP450 inhibitor, which is a compound that reduces the activity of the CYP450 enzyme, may increase the drug exposure, resulting in increased therapeutic plasma levels of those drugs over time. Consequently, there is an unmet need for methods for administering azetukalner to a patient in need thereof, wherein the patient is also being treated with another substance which may interact with the said drug, such as a CYP450 inhibitor.

### SUMMARY

The present disclosure describes certain methods utilizing N-[4-(6-Fluoro-3,4-dihydro-1H-isoquinolin-2-yl)-2,6-dimethylphenyl]-3,3-dimethylbutanamide (herein referred to as “Compound A” or by its international nonproprietary name (“INN”) “azetukalner”), or a pharmaceutically acceptable salt thereof.

In one aspect, the present disclosure describes a method of treating a disease, disorder, or condition in a human in need thereof, wherein the human is being administered a cytochrome P450 3A4 (CYP3A4) inhibitor, the method comprising (a) administering to the human a therapeutically effective amount of azetukalner. and (b) discontinuing the administration of the CYP3A4 inhibitor.

In another aspect, the present disclosure describes a method of treating a disease, disorder, or condition in a human in need thereof, wherein the human is being administered a cytochrome P450 3A4 (CYP3A4) inhibitor, the method comprising (a) discontinuing the administration of the CYP3A4 inhibitor, and (b) administering to the human a therapeutically effective amount of azetukalner, thereby avoiding the use of the CYP3A4 inhibitor in combination with azetukalner.

In a further aspect, the present disclosure describes a method of treating a disease, disorder, or condition in a human in need thereof, the method comprising (a) administering to the human a therapeutically effective amount of azetukalner, (b) subsequently determining that the human is being administered a cytochrome P450 3A4 (CYP3A4) inhibitor, and (c) discontinuing the administration of the CYP3A4 inhibitor.

In one aspect, the present disclosure describes an improvement in a method of treating a disease, disorder, or condition in a human in need thereof, the method comprising administering to the human a therapeutically effective amount of azetukalner, wherein the human is being administered a cytochrome P450 3A4 (CYP3A4) inhibitor, the improvement comprising discontinuing the administration of the CYP3A4 inhibitor.

In another aspect, the present disclosure describes an improvement in a method of treating a disease, disorder, or condition in a human in need thereof, wherein the human is being administered a cytochrome P450 3A4 (CYP3A4) inhibitor, the improvement comprising (a) discontinuing the administration of the CYP3A4 inhibitor, and (b) administering to the human a therapeutically effective amount of azetukalner. thereby avoiding the use of the CYP3A4 inhibitor in combination with azetukalner.

In a further aspect, the present disclosure describes an improvement in a method of treating a disease, disorder, or condition in a human in need thereof, the improvement comprising (a) administering to the human a therapeutically effective amount of azetukalner, (b) subsequently determining that the human is being administered a cytochrome P450 3A4 (CYP3A4) inhibitor, and (c) discontinuing the administration of the CYP3A4 inhibitor.

In yet another aspect, the present disclosure describes a method of treating a disease, disorder, or condition in a human in need thereof, wherein the human is being administered a cytochrome P450 3A4 (CYP3A4) inhibitor, the method comprising (a) administering to the human a therapeutically effective amount of azetukalner, and (b) reducing the dose of the CYP3A4 inhibitor being administered to the human.

In one aspect, the present disclosure describes a method of treating a disease, disorder, or condition in a human in need thereof, wherein the human is being administered a cytochrome P450 3A4 (CYP3A4) inhibitor, the method comprising (a) reducing the dose of the CYP3A4 inhibitor being administered to the human, and (b) administering to the human a therapeutically effective amount of azetukalner.

In another aspect, the present disclosure describes a method of treating a disease, disorder, or condition in a human in need thereof, the method comprising (a) administering to the human a therapeutically effective amount of azetukalner, (b) subsequently determining that the human is being administered a cytochrome P450 3A4 (CYP3A4) inhibitor, and (c) reducing the dosage of the CYP3A4 inhibitor being administered to the human.

In a further aspect, the present disclosure describes an improvement in a method of treating a disease, disorder, or condition in a human in need thereof, the method comprising administering to the human a therapeutically effective amount of azetukalner, wherein the human is being administered a cytochrome P450 3A4 (CYP3A4) inhibitor, the improvement comprising reducing the dose of the CYP3A4 inhibitor being administered to the human.

In yet another aspect, the present disclosure describes an improvement in a method of treating a disease, disorder, or condition in a human in need thereof, wherein the human is being administered a cytochrome P450 3A4 (CYP3A4) inhibitor, the improvement comprising (a) administering to the human a therapeutically effective amount of azetukalner, and (b) reducing the dose of the CYP3A4 inhibitor being administered to the human.

In one aspect, the present disclosure describes an improvement in a method of treating a disease, disorder, or condition in a human in need thereof, wherein the human is being administered a cytochrome P450 3A4 (CYP3A4) inhibitor, the improvement comprising, (a) reducing the dose of the CYP3A4 inhibitor being administered to the human, and (b) administering to the human a therapeutically effective amount of azetukalner.

In another aspect, the present disclosure describes an improvement in a method of treating a disease, disorder, or condition in a human in need thereof, the improvement comprising (a) administering to the human a therapeutically effective amount of azetukalner, (b) subsequently determining that the human is being administered a cytochrome P450 3A4 (CYP3A4) inhibitor, and (c) reducing the dose of the CYP3A4 inhibitor being administered to the human.

In a further aspect, the present disclosure describes a method of treating a disease, disorder, or condition in a human in need thereof, wherein the human is being administered a cytochrome P450 3A4 (CYP3A4) inhibitor, the method comprising (a) administering to the human a dose of azetukalner that is reduced compared to the dose of azetukalner that would be administered to a human not being administered a CYP3A4 inhibitor.

In yet another aspect, the present disclosure describes a method of treating a disease, disorder, or condition in a human in need thereof, wherein the human is being administered a cytochrome P450 3A4 (CYP3A4) inhibitor, the method comprising (a) administering to the human a therapeutically effective amount of azetukalner, and (b) reducing the dose of azetukalner being administered to the human.

In one aspect, the present disclosure describes a method of treating a disease, disorder, or condition in a human in need thereof, the method comprising (a) administering to the human a therapeutically effective amount of azetukalner, (b) subsequently determining that the human is being administered a cytochrome P450 3A4 (CYP3A4) inhibitor, and (c) reducing the dose of azetukalner being administered to the human.

In another aspect, the present disclosure describes an improvement in a method of treating a disease, disorder, or condition in a human in need thereof, the method comprising administering to the human a therapeutically effective amount of azetukalner, wherein the human is being administered a cytochrome P450 3A4 (CYP3A4) inhibitor, the improvement comprising reducing the dose of azetukalner being administered to the human.

In a further aspect, the present disclosure describes an improvement in a method of treating a disease, disorder, or condition in a human in need thereof, the method comprising administering to the human a therapeutically effective amount of azetukalner, wherein the human is being administered a cytochrome P450 3A4 (CYP3A4) inhibitor, the improvement comprising administering to the human a dose of azetukalner that is reduced compared to the dose of azetukalner that would be administered to a human not being administered a CYP3A4 inhibitor.

In yet another aspect, the present disclosure describes an improvement in a method of treating a disease, disorder, or condition in a human in need thereof, the method comprising administering to the human a therapeutically effective amount of azetukalner, wherein the human is being administered a cytochrome P450 3A4 (CYP3A4) inhibitor, the improvement comprising (a) administering to the human a therapeutically effective amount of azetukalner, (b) subsequently determining that the human is being administered a cytochrome P450 3A4 (CYP3A4) inhibitor, and (c) reducing the dose of azetukalner being administered to the human.

In one aspect, the present disclosure describes a method of treating a disease, disorder, or condition in a human in need thereof, wherein the human is a poor cytochrome P450 3A4 (CYP3A4) metabolizer, the method comprising administering to the human a dose of azetukalner that is reduced compared to the dose of azetukalner that would be administered to a human who is not a poor CYP3A4 metabolizer.

In another aspect, the present disclosure describes a method of treating a disease, disorder, or condition in a human in need thereof, wherein the human is a poor cytochrome P450 3A4 (CYP3A4) metabolizer, the method comprising (a) administering to the human a therapeutically effective amount of azetukalner; an (b) reducing the dose of azetukalner being administered to the human.

In a further aspect, the present disclosure describes a method of treating a disease, disorder, or condition in a human in need thereof, the method comprising (a) administering to the human a therapeutically effective amount of azetukalner, (b) subsequently determining that the human is a poor cytochrome P450 3A4 (CYP3A4) metabolizer, and (c) reducing the dose of azetukalner being administered to the human.

In yet another aspect, the present disclosure describes a method of treating a disease, disorder, or condition in a human in need thereof, the method comprising (a) determining if the human is a poor cytochrome P450 3A4 (CYP3A4) metabolizer, and (b) if the human is a poor CYP3A4 metabolizer, administering to the human a dose of azetukalner that is reduced compared to the dose of azetukalner that would be administered to a human who is not a poor CYP3A4 metabolizer.

In one aspect, the present disclosure describes method of treating a disease, disorder, or condition in a human in need thereof, wherein the human is being administered a therapeutically effective amount of azetukalner, the method comprising (a) determining if the human is a poor cytochrome P450 3A4 (CYP3A4) metabolizer, and (b) if the human is a poor CYP3A4 metabolizer, reducing the dose of azetukalner being administered to the human.

In another aspect, the present disclosure describes an improvement in a method of treating a disease, disorder, or condition in a human in need thereof, the method comprising administering to the human a therapeutically effective amount of azetukalner, wherein the human is a poor cytochrome P450 3A4 (CYP3A4) metabolizer, the improvement comprising administering to the human a dose of azetukalner that is reduced compared to the dose of azetukalner that would be administered to a human who is not a poor CYP3A4 metabolizer.

In a further aspect, the present disclosure describes an improvement in a method of treating a disease, disorder, or condition in a human in need thereof, the method comprising administering to the human a therapeutically effective amount of azetukalner, wherein the human is a poor cytochrome P450 3A4 (CYP3A4) metabolizer, the improvement comprising reducing the dose of azetukalner being administered to the human.

In yet another aspect, the present disclosure describes an improvement in a method of treating a disease, disorder, or condition in a human in need thereof, the improvement comprising (a) administering to the human a therapeutically effective amount of azetukalner, (b) subsequently determining that the human is a poor cytochrome P450 3A4 (CYP3A4) metabolizer, and (c) reducing the dose of azetukalner being administered to the human.

In one aspect, the present disclosure describes an improvement in a method of treating a disease, disorder, or condition in a human in need thereof, the improvement comprising (a) determining if the human is a poor cytochrome P450 3A4 (CYP3A4) metabolizer, and (b) if the human is a poor CYP3A4 metabolizer, administering to the human a dose of azetukalner that is reduced compared to the dose of azetukalner that would be administered to a human who is not a poor CYP3A4 metabolizer.

In another aspect, the present disclosure describes an improvement in a method of treating a disease, disorder, or condition in a human in need thereof, wherein the human is being administered a therapeutically effective amount of azetukalner, the improvement comprising (a) determining if the human is a poor cytochrome P450 3A4 (CYP3A4) metabolizer, and (b) if the human is a poor CYP3A4 metabolizer, reducing the dose of azetukalner being administered to the human.

The details of certain embodiments of the disclosure are set forth in the Detailed Description, as described below. Other embodiments of the disclosure will be apparent from the Definitions, Examples, Abstract, and Claims.

### Definitions

As used in the specification and appended claims, unless specified to the contrary, the following terms and abbreviations have the meaning indicated:

“Compound A” or “azetukalner” refers to the compound having the following formula:

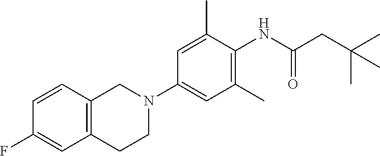 [SMILES: Cc1cc(N2CCc3cc(F)ccc3C2)cc(C)c1NC(=O)CC(C)(C)C]

and having a chemical name of N-[4-(6-fluoro-3,4-dihydro-1H-isoquinolin-2-yl)-2,6-dimethylphenyl]-3,3-dimethylbutanamide. Azetukalner is a potent small molecule Kv7 potassium channel opener currently being developed for the treatment of epilepsy, major depressive disorder, and potentially other neurological disorders. Preparation of Azetukalner and its use as a potassium channel (e.g., Kv7.2/Kv7.3 (KCNQ2/3)) opener is disclosed in U.S. Pat. Nos. 8,293,911, 8,993,593, and 11,135,214. Azetukalner is different from most known AED's in that it potentiates and enhances opening of the voltage-gated potassium channels (e.g., Kv7.2 and Kv7.3 (Kv7.2/Kv7.3)), which are important in controlling neuronal excitability. Azetukalner is used in the methods described herein. Azetukalner may be provided as a solid amorphous form or in one or more crystalline forms described in U.S. Pat. No. 11,091,441, which is incorporated herein by reference in its entirety.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease, disorder, or condition.

The term “administer,” “administering,” or “administration” refers to implanting, absorbing, ingesting, injecting, inhaling, providing or otherwise introducing a compound described herein, or a composition thereof, in, to or on a subject. In certain embodiments, “administration” refers to oral administration. In certain embodiments, “administration” refers to intravenous administration.

The terms “systemic administration” and “systemically administering” and the like refer to the administration of a compound such that it enters a subject's bloodstream. Thus, systemically administered compounds may affect the entire body (e.g. the compound may be subject to metabolism and other like processes). Systemic administration may include, for example, intravenous, intraperitoneal, intra-arthrodial (within joints) intramuscular or subcutaneous injections, or inhalation, transdermal or oral administration.

The terms “orally administering” and “oral administration” and the like refer to the administration of a compound by taking the compound into the body via the mouth. Oral administration may include, for example, oral, buccal, enteral, or intragastric administration. In some embodiments, oral administration may be limited to administration by swallowing.

The term “co-administration” refers to implanting, absorbing, ingesting, injecting, inhaling, providing or otherwise introducing a compound described herein, or a composition thereof, in, to or on a subject with additional agents.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen).

Treatment may also be continued after symptoms have resolved, for example, to delay or prevent recurrence.

The terms “condition,” “disease,” and “disorder” are used interchangeably.

As used herein “co-administration” and “concomitant treatment” are used interchangeable (e.g., “systemic co-administration” and “concomitant systemic treatment” are used interchangeably).

An “effective amount” of a compound described herein refers to an amount sufficient to elicit the desired biological response. An effective amount of a compound described herein may vary depending on such factors as the desired biological endpoint, severity of side effects, disease, or disorder, the identity, pharmacokinetics, and pharmacodynamics of the particular compound, the condition being treated, the mode, route, and desired or required frequency of administration, the species, age and health or general condition of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses. In certain embodiments, an effective amount is an amount sufficient for potentiating a Kv7 potassium channel (e.g., in a subject or in a cell in vitro).

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent. In certain embodiments, a therapeutically effective amount is an amount sufficient for treating a disease, disorder, or condition (e.g., a disease, disorder, or condition associated with Kv7 potassium channel dysfunction) in a subject. In certain embodiments, a therapeutically effective amount is an amount sufficient for potentiating a Kv7 potassium channel in a subject.

“Seizure disorders” refers to seizures and disorders associated with seizures such as focal onset epilepsy, photosensitive epilepsy, seizures associated with self-induced syncope, intractable epilepsy, seizures associated with Angelman syndrome, benign rolandic epilepsy, CDKL5 disorder, childhood and juvenile absence epilepsy, Dravet syndrome, frontal lobe epilepsy, Glut1 deficiency syndrome, seizures associated with hypothalamic hamartoma, infantile spasms/West's syndrome, juvenile myoclonic epilepsy, Landau-Kleffner syndrome, Lennox-Gastaut syndrome (LGS), epilepsy with myoclonic-absences. Ohtahara syndrome, Panayiotopoulos syndrome, PCDH19 epilepsy, progressive myoclonic epilepsies, seizures associated with Rasmussen's syndrome, ring chromosome 20 syndrome, reflex epilepsies, temporal lobe epilepsy, Lafora progressive myoclonus epilepsy, seizures associated with neurocutaneous syndromes, seizures associated with tuberous sclerosis complex, early infantile epileptic encephalopathy, early onset epileptic encephalopathy, generalized epilepsy, generalized epilepsy with febrile seizures+, seizures associated with Rett syndrome, seizures associated with multiple sclerosis, Alzheimer's disease, seizures associated with autism, seizures associated with ataxia, seizures associated with hypotonia, paroxysmal dyskinesia, generalized onset seizures, focal onset seizures, or primary generalized tonic-clonic seizures, or a combination thereof. In certain embodiments, the seizure disorder is focal onset epilepsy, also known as partial onset (focal) epilepsy. In certain embodiments, the seizure disorder is primary generalized tonic-clonic seizures.

“Voltage-gated potassium channels” (VGKCs) are transmembrane channels specific for potassium and sensitive to voltage changes in a cell's membrane potential. During action potentials. they play an important role in returning the depolarized cell to a resting state. In VGKCs, alpha subunits form the actual conductance pore. Based on sequence homology of the hydrophobic transmembrane cores, the alpha subunits of voltage-gated potassium channels are grouped into 12 families (Kv1-12), of which Kv7 is one family. The Kv7 family of voltage-gated potassium channels consists of five members (Kv7.1-7.5) which are encoded for by the KCNQ1-5 genes, respectively. Of the five, Kv7.2/Kv7.3 is the active heterotetramer that is the main active Kv7 current in neurons (M-current). A Kv7 potassium channel can be a member selected from Kv7.1, Kv7.2, Kv7.3, Kv7.4, and/or Kv7.5.

“Potassium channel potentiator” and “potassium channel opener” are used interchangeably and refer to an agent that restores, enhances, or increases the activity of a potassium channel (e.g., voltage-gated potassium channel, e.g., Kv7 potassium channel), for example, by facilitating ion transmission through the potassium channel. “Potentiating,” “potentiation,” and the like, for the purposes of this disclosure, refer to restoring, enhancing, or increasing the activity or effect of a potassium channel.

### DETAILED DESCRIPTION OF CERTAIN EMBODIMENTS

Azetukalner is mostly metabolized by the liver, and cytochrome P450 3A4 (CYP3A4) is the main enzyme involved in its metabolism, while other CYP isozymes play a very limited or no role. Effects of azetuklaner with CYP3A4 inhibitors or CYP3A4 inducers have been reported, for example, CYP3A4 inhibitors have been shown to increase the drug exposure, while CYP3A4 inducers were shown to decrease the drug exposure. See Bialer et al. Epilepsia 2022, 63(11), 2883-2910, at pg. 22, left column; see also Wang et al. Epilepsy 2022, 18(1), 2-4, at pg. 3, left column. However, it was understood that additional evidence was needed to understand the clinical relevance of these potential interactions. See Lattanzi et al. Exp. Rev. Clin. Pharmacol. 2024, 7(5-6), 423-432, 430.

As described herein, the inventors unexpectedly discovered during the course of drug-drug interaction studies that co-administration of azetukalner with a strong CYP3A4 inhibitor (itraconazole) increased exposure of azetukalner, with an approximately ˜13% increase in Cₘₐₓ and a 2-fold increase in AUC_{0-∞}. Based on additional preliminary pharmacokinetic modeling of azetukalner with prolonged systemic administration with a strong CYP3A4 inhibitor, the inventors discovered the methods of administering azetukalner disclosed herein.

For example, the inventors discovered that concomitant administration with a CYP3A4 inhibitor is acceptable for short-term co-administration periods, allowing for patients to continue taking azetukalner uninterrupted for a specified period, but discontinuing co-administration of the CYP3A4 inhibitor thereafter to preclude long-term co-administration periods. Such methods unexpectedly allow for continued administration of azetukalner without adverse impact on its efficacy or side effects, while permitting co-administration of a strong CYP3A4 inhibitor for the treatment of another condition, for a limited time. This discovery is particularly useful, e.g., for patients being administered azetukalner for a chronic condition (e.g., a seizure disorder) but with the need to be co-administered a CYP3A4 inhibitor for a limited time (e.g., administration of ritonavir, as in PAXLOVID™ (nirmatrelvir and ritonavir), for the concomitant transient treatment of a COVID-19 infection. See PAXLOVID™ (nirmatrelvir and ritonavir), Full Prescribing Information, May 2023 (providing a recommended twice daily dosing for 5 days)). Thus, in addition to the disease, disorder or condition as described herein, the subject may also have a comorbidity that is susceptible to treatment with a CYP3A4 inhibitor, such as a COVID-19 infection which may be treated (as described above) with nirmatrelvir and ritonavir.

As described above, the co-administration of azetukalner by CYP3A4 inhibitors or inducers can alter its pharmacokinetic profile, resulting in high or low levels of azetukalner in the body and can lead to side effects or ineffective therapy. To manage this potential drug-drug interaction, the inventors have developed methods for administering azetukalner to a subject (e.g., a human) that is also being systematically administered a CYP3A4 inhibitor. Accordingly, certain methods disclosed herein provide for modified dosing, for example, by reducing the amount of azetukalner or CYP3A4 inhibitor being co-administered to the subject, discontinuing the administration of azetukalner or CYP3A4 inhibitor in favor of the other drug being administered, or by prohibiting co-administration of azetukalner with a CYP3A4 inhibitor altogether.

The present disclosure further contemplates methods for administering azetukalner to patients that may have difficulty metabolizing the drug. For example, subjects considered to be “poor CYP3A4 metabolizers” (e.g., subjects having little or no CYP3A4 function) may be at risk for adverse drug reactions due to increased drug exposure. Accordingly, the inventors developed methods disclosed herein that provide for modified dosing, for example, by reducing the amount of azetukalner or CYP3A4 inhibitor being co-administered to the subject, discontinuing the administration of azetukalner or CYP3A4 inhibitor in favor of the other drug being administered, or by prohibiting co-administration of azetukalner with a CYP3A4 inhibitor altogether.

In one embodiment, the present disclosure describes a method of treating a disease, disorder, or condition in a human in need thereof, wherein the human is being administered a cytochrome P450 3A4 (CYP3A4) inhibitor, the method comprising (a) administering to the human a therapeutically effective amount of azetukalner, and (b) discontinuing the administration of the CYP3A4 inhibitor.

In another embodiment, the present disclosure describes a method of treating a disease, disorder, or condition in a human in need thereof, wherein the human is being administered a cytochrome P450 3A4 (CYP3A4) inhibitor, the method comprising (a) discontinuing the administration of the CYP3A4 inhibitor, and (b) administering to the human a therapeutically effective amount of azetukalner, thereby avoiding the use of the CYP3A4 inhibitor in combination with azetukalner.

In a further embodiment, the present disclosure describes a method of treating a disease, disorder, or condition in a human in need thereof, the method comprising (a) administering to the human a therapeutically effective amount of azetukalner, (b) subsequently determining that the human is being administered a cytochrome P450 3A4 (CYP3A4) inhibitor, and (c) discontinuing the administration of the CYP3A4 inhibitor.

In one embodiment, the present disclosure describes an improvement in a method of treating a disease, disorder, or condition in a human in need thereof, the method comprising administering to the human a therapeutically effective amount of azetukalner, wherein the human is being administered a cytochrome P450 3A4 (CYP3A4) inhibitor, the improvement comprising discontinuing the administration of the CYP3A4 inhibitor.

In another embodiment, the present disclosure describes an improvement in a method of treating a disease, disorder, or condition in a human in need thereof, wherein the human is being administered a cytochrome P450 3A4 (CYP3A4) inhibitor, the improvement comprising (a) discontinuing the administration of the CYP3A4 inhibitor, and (b) administering to the human a therapeutically effective amount of azetukalner, thereby avoiding the use of the CYP3A4 inhibitor in combination with azetukalner.

In a further embodiment, the present disclosure describes an improvement in a method of treating a disease, disorder, or condition in a human in need thereof, the improvement comprising (a) administering to the human a therapeutically effective amount of azetukalner, (b) subsequently determining that the human is being administered a cytochrome P450 3A4 (CYP3A4) inhibitor, and (c) discontinuing the administration of the CYP3A4 inhibitor.

In yet another embodiment, the present disclosure describes a method of treating a disease, disorder, or condition in a human in need thereof, wherein the human is being administered a cytochrome P450 3A4 (CYP3A4) inhibitor, the method comprising (a) administering to the human a therapeutically effective amount of azetukalner, and (b) reducing the dose of the CYP3A4 inhibitor being administered to the human.

In one embodiment, the present disclosure describes a method of treating a disease, disorder, or condition in a human in need thereof, wherein the human is being administered a cytochrome P450 3A4 (CYP3A4) inhibitor, the method comprising (a) reducing the dose of the CYP3A4 inhibitor being administered to the human, and (b) administering to the human a therapeutically effective amount of azetukalner.

In another embodiment, the present disclosure describes a method of treating a disease, disorder, or condition in a human in need thereof, the method comprising (a) administering to the human a therapeutically effective amount of azetukalner, (b) subsequently determining that the human is being administered a cytochrome P450 3A4 (CYP3A4) inhibitor, and (c) reducing the dosage of the CYP3A4 inhibitor being administered to the human.

In a further embodiment, the present disclosure describes an improvement in a method of treating a disease, disorder, or condition in a human in need thereof, the method comprising administering to the human a therapeutically effective amount of azetukalner, wherein the human is being administered a cytochrome P450 3A4 (CYP3A4) inhibitor, the improvement comprising reducing the dose of the CYP3A4 inhibitor being administered to the human.

In yet another embodiment, the present disclosure describes an improvement in a method of treating a disease, disorder, or condition in a human in need thereof, wherein the human is being administered a cytochrome P450 3A4 (CYP3A4) inhibitor, the improvement comprising (a) administering to the human a therapeutically effective amount of azetukalner, and (b) reducing the dose of the CYP3A4 inhibitor being administered to the human.

In one embodiment, the present disclosure describes an improvement in a method of treating a disease, disorder, or condition in a human in need thereof, wherein the human is being administered a cytochrome P450 3A4 (CYP3A4) inhibitor, the improvement comprising, (a) reducing the dose of the CYP3A4 inhibitor being administered to the human, and (b) administering to the human a therapeutically effective amount of azetukalner.

In another embodiment, the present disclosure describes an improvement in a method of treating a disease, disorder, or condition in a human in need thereof, the improvement comprising (a) administering to the human a therapeutically effective amount of azetukalner, (b) subsequently determining that the human is being administered a cytochrome P450 3A4 (CYP3A4) inhibitor, and (c) reducing the dose of the CYP3A4 inhibitor being administered to the human.

In a further embodiment, the present disclosure describes a method of treating a disease, disorder, or condition in a human in need thereof, wherein the human is being administered a cytochrome P450 3A4 (CYP3A4) inhibitor, the method comprising (a) administering to the human a dose of azetukalner that is reduced compared to the dose of azetukalner that would be administered to a human not being administered a CYP3A4 inhibitor.

In yet another embodiment, the present disclosure describes a method of treating a disease, disorder, or condition in a human in need thereof, wherein the human is being administered a cytochrome P450 3A4 (CYP3A4) inhibitor, the method comprising (a) administering to the human a therapeutically effective amount of azetukalner, and (b) reducing the dose of azetukalner being administered to the human.

In one embodiment, the present disclosure describes a method of treating a disease, disorder, or condition in a human in need thereof, the method comprising (a) administering to the human a therapeutically effective amount of azetukalner, (b) subsequently determining that the human is being administered a cytochrome P450 3A4 (CYP3A4) inhibitor, and (c) reducing the dose of azetukalner being administered to the human.

In another embodiment, the present disclosure describes an improvement in a method of treating a disease, disorder, or condition in a human in need thereof, the method comprising administering to the human a therapeutically effective amount of azetukalner, wherein the human is being administered a cytochrome P450 3A4 (CYP3A4) inhibitor, the improvement comprising reducing the dose of azetukalner being administered to the human.

In a further embodiment, the present disclosure describes an improvement in a method of treating a disease, disorder, or condition in a human in need thereof, the method comprising administering to the human a therapeutically effective amount of azetukalner, wherein the human is being administered a cytochrome P450 3A4 (CYP3A4) inhibitor, the improvement comprising administering to the human a dose of azetukalner that is reduced compared to the dose of azetukalner that would be administered to a human not being administered a CYP3A4 inhibitor.

In yet another embodiment, the present disclosure describes an improvement in a method of treating a disease, disorder, or condition in a human in need thereof, the method comprising administering to the human a therapeutically effective amount of azetukalner, wherein the human is being administered a cytochrome P450 3A4 (CYP3A4) inhibitor, the improvement comprising (a) administering to the human a therapeutically effective amount of azetukalner, (b) subsequently determining that the human is being administered a cytochrome P450 3A4 (CYP3A4) inhibitor, and (c) reducing the dose of azetukalner being administered to the human.

In one embodiment, the present disclosure describes a method of treating a disease, disorder, or condition in a human in need thereof, wherein the human is a poor cytochrome P450 3A4 (CYP3A4) metabolizer, the method comprising administering to the human a dose of azetukalner that is reduced compared to the dose of azetukalner that would be administered to a human who is not a poor CYP3A4 metabolizer.

In another embodiment, the present disclosure describes a method of treating a disease, disorder, or condition in a human in need thereof, wherein the human is a poor cytochrome P450 3A4 (CYP3A4) metabolizer, the method comprising (a) administering to the human a therapeutically effective amount of azetukalner; and (b) reducing the dose of azetukalner being administered to the human.

In a further embodiment, the present disclosure describes a method of treating a disease, disorder, or condition in a human in need thereof, the method comprising (a) administering to the human a therapeutically effective amount of azetukalner, (b) subsequently determining that the human is a poor cytochrome P450 3A4 (CYP3A4) metabolizer, and (c) reducing the dose of azetukalner being administered to the human.

In yet another embodiment, the present disclosure describes a method of treating a disease, disorder, or condition in a human in need thereof, the method comprising (a) determining if the human is a poor cytochrome P450 3A4 (CYP3A4) metabolizer, and (b) if the human is a poor CYP3A4 metabolizer, administering to the human a dose of azetukalner that is reduced compared to the dose of azetukalner that would be administered to a human who is not a poor CYP3A4 metabolizer.

In one embodiment, the present disclosure describes method of treating a disease, disorder, or condition in a human in need thereof, wherein the human is being administered a therapeutically effective amount of azetukalner, the method comprising (a) determining if the human is a poor cytochrome P450 3A4 (CYP3A4) metabolizer, and (b) if the human is a poor CYP3A4 metabolizer, reducing the dose of azetukalner being administered to the human.

In another embodiment, the present disclosure describes an improvement in a method of treating a disease, disorder, or condition in a human in need thereof, the method comprising administering to the human a therapeutically effective amount of azetukalner, wherein the human is a poor cytochrome P450 3A4 (CYP3A4) metabolizer, the improvement comprising administering to the human a dose of azetukalner that is reduced compared to the dose of azetukalner that would be administered to a human who is not a poor CYP3A4 metabolizer.

In a further embodiment, the present disclosure describes an improvement in a method of treating a disease, disorder, or condition in a human in need thereof, the method comprising administering to the human a therapeutically effective amount of azetukalner, wherein the human is a poor cytochrome P450 3A4 (CYP3A4) metabolizer, the improvement comprising reducing the dose of azetukalner being administered to the human.

In yet another embodiment, the present disclosure describes an improvement in a method of treating a disease, disorder, or condition in a human in need thereof, the improvement comprising (a) administering to the human a therapeutically effective amount of azetukalner, (b) subsequently determining that the human is a poor cytochrome P450 3A4 (CYP3A4) metabolizer, and (c) reducing the dose of azetukalner being administered to the human.

In one embodiment, the present disclosure describes an improvement in a method of treating a disease, disorder, or condition in a human in need thereof, the improvement comprising (a) determining if the human is a poor cytochrome P450 3A4 (CYP3A4) metabolizer, and (b) if the human is a poor CYP3A4 metabolizer, administering to the human a dose of azetukalner that is reduced compared to the dose of azetukalner that would be administered to a human who is not a poor CYP3A4 metabolizer.

In another embodiment, the present disclosure describes an improvement in a method of treating a disease, disorder, or condition in a human in need thereof, wherein the human is being administered a therapeutically effective amount of azetukalner, the improvement comprising (a) determining if the human is a poor cytochrome P450 3A4 (CYP3A4) metabolizer, and (b) if the human is a poor CYP3A4 metabolizer, reducing the dose of azetukalner being administered to the human.

In one embodiment, the present disclosure describes a use of azetukalner in treating a disease, disorder, or condition in a human in need thereof, wherein the human is being administered a cytochrome P450 3A4 (CYP3A4) inhibitor, the use comprising (a) administering to the human a therapeutically effective amount of azetukalner, and (b) discontinuing the administration of the CYP3A4 inhibitor.

In another embodiment, the present disclosure describes a use of azetukalner in treating a disease, disorder, or condition in a human in need thereof, wherein the human is being administered a cytochrome P450 3A4 (CYP3A4) inhibitor, the use comprising (a) discontinuing the administration of the CYP3A4 inhibitor, and (b) administering to the human a therapeutically effective amount of azetukalner, thereby avoiding the use of the CYP3A4 inhibitor in combination with azetukalner.

In a further embodiment, the present disclosure describes a use of azetukalner in treating a disease, disorder, or condition in a human in need thereof, the use comprising (a) administering to the human a therapeutically effective amount of azetukalner, (b) subsequently determining that the human is being administered a cytochrome P450 3A4 (CYP3A4) inhibitor, and (c) discontinuing the administration of the CYP3A4 inhibitor.

In yet another embodiment, the present disclosure describes a use of azetukalner in treating a disease, disorder, or condition in a human in need thereof, wherein the human is being administered a cytochrome P450 3A4 (CYP3A4) inhibitor, the use comprising (a) administering to the human a therapeutically effective amount of azetukalner, and (b) reducing the dose of the CYP3A4 inhibitor being administered to the human.

In one embodiment, the present disclosure describes a use of azetukalner in treating a disease, disorder, or condition in a human in need thereof, wherein the human is being administered a cytochrome P450 3A4 (CYP3A4) inhibitor, the use comprising (a) reducing the dose of the CYP3A4 inhibitor being administered to the human, and (b) administering to the human a therapeutically effective amount of azetukalner.

In another embodiment, the present disclosure describes a use of azetukalner in treating a disease, disorder, or condition in a human in need thereof, the use comprising (a) administering to the human a therapeutically effective amount of azetukalner, (b) subsequently determining that the human is being administered a cytochrome P450 3A4 (CYP3A4) inhibitor, and (c) reducing the dosage of the CYP3A4 inhibitor being administered to the human.

In a further embodiment, the present disclosure describes a use of azetukalner in treating a disease, disorder, or condition in a human in need thereof, wherein the human is being administered a cytochrome P450 3A4 (CYP3A4) inhibitor, the use comprising (a) administering to the human a dose of azetukalner that is reduced compared to the dose of azetukalner that would be administered to a human not being administered a CYP3A4 inhibitor.

In yet another embodiment, the present disclosure describes a use of azetukalner in treating a disease, disorder, or condition in a human in need thereof, wherein the human is being administered a cytochrome P450 3A4 (CYP3A4) inhibitor, the use comprising (a) administering to the human a therapeutically effective amount of azetukalner, and (b) reducing the dose of azetukalner being administered to the human.

In one embodiment, the present disclosure describes a use of azetukalner in treating a disease, disorder, or condition in a human in need thereof, the use comprising (a) administering to the human a therapeutically effective amount of azetukalner, (b) subsequently determining that the human is being administered a cytochrome P450 3A4 (CYP3A4) inhibitor, and (c) reducing the dose of azetukalner being administered to the human.

In one embodiment, the present disclosure describes a use of azetukalner in treating a disease, disorder, or condition in a human in need thereof, wherein the human is a poor cytochrome P450 3A4 (CYP3A4) metabolizer, the use comprising administering to the human a dose of azetukalner that is reduced compared to the dose of azetukalner that would be administered to a human who is not a poor CYP3A4 metabolizer.

In another embodiment, the present disclosure describes a use of azetukalner in treating a disease, disorder, or condition in a human in need thereof, wherein the human is a poor cytochrome P450 3A4 (CYP3A4) metabolizer, the use comprising (a) administering to the human a therapeutically effective amount of azetukalner; and (b) reducing the dose of azetukalner being administered to the human.

In a further embodiment, the present disclosure describes a use of azetukalner in treating a disease, disorder, or condition in a human in need thereof, the use comprising (a) administering to the human a therapeutically effective amount of azetukalner, (b) subsequently determining that the human is a poor cytochrome P450 3A4 (CYP3A4) metabolizer, and (c) reducing the dose of azetukalner being administered to the human.

In yet another embodiment, the present disclosure describes a use of azetukalner in treating a disease, disorder, or condition in a human in need thereof, the use comprising (a) determining if the human is a poor cytochrome P450 3A4 (CYP3A4) metabolizer, and (b) if the human is a poor CYP3A4 metabolizer, administering to the human a dose of azetukalner that is reduced compared to the dose of azetukalner that would be administered to a human who is not a poor CYP3A4 metabolizer.

In one embodiment, the present disclosure describes a use of azetukalner in treating a disease, disorder, or condition in a human in need thereof, wherein the human is being administered a therapeutically effective amount of azetukalner, the use comprising (a) determining if the human is a poor cytochrome P450 3A4 (CYP3A4) metabolizer, and (b) if the human is a poor CYP3A4 metabolizer, reducing the dose of azetukalner being administered to the human.

In one embodiment, the present disclosure describes azetukalner for use in treating a disease, disorder, or condition in a human in need thereof, wherein the human is being administered a cytochrome P450 3A4 (CYP3A4) inhibitor, the use comprising (a) administering to the human a therapeutically effective amount of azetukalner, and (b) discontinuing the administration of the CYP3A4 inhibitor.

In another embodiment, the present disclosure describes azetukalner for use in treating a disease, disorder, or condition in a human in need thereof, wherein the human is being administered a cytochrome P450 3A4 (CYP3A4) inhibitor, the use comprising (a) discontinuing the administration of the CYP3A4 inhibitor, and (b) administering to the human a therapeutically effective amount of azetukalner, thereby avoiding the use of the CYP3A4 inhibitor in combination with azetukalner.

In a further embodiment, the present disclosure describes azetukalner for use in treating a disease, disorder, or condition in a human in need thereof, the use comprising (a) administering to the human a therapeutically effective amount of azetukalner, (b) subsequently determining that the human is being administered a cytochrome P450 3A4 (CYP3A4) inhibitor, and (c) discontinuing the administration of the CYP3A4 inhibitor.

In yet another embodiment, the present disclosure describes azetukalner for use in treating a disease, disorder, or condition in a human in need thereof, wherein the human is being administered a cytochrome P450 3A4 (CYP3A4) inhibitor, the use comprising (a) administering to the human a therapeutically effective amount of azetukalner, and (b) reducing the dose of the CYP3A4 inhibitor being administered to the human.

In one embodiment, the present disclosure describes azetukalner for use in treating a disease, disorder, or condition in a human in need thereof, wherein the human is being administered a cytochrome P450 3A4 (CYP3A4) inhibitor, the use comprising (a) reducing the dose of the CYP3A4 inhibitor being administered to the human, and (b) administering to the human a therapeutically effective amount of azetukalner.

In another embodiment, the present disclosure describes azetukalner for use in treating a disease, disorder, or condition in a human in need thereof, the use comprising (a) administering to the human a therapeutically effective amount of azetukalner, (b) subsequently determining that the human is being administered a cytochrome P450 3A4 (CYP3A4) inhibitor, and (c) reducing the dosage of the CYP3A4 inhibitor being administered to the human.

In a further embodiment, the present disclosure describes azetukalner for use in treating a disease, disorder, or condition in a human in need thereof, wherein the human is being administered a cytochrome P450 3A4 (CYP3A4) inhibitor, the use comprising (a) administering to the human a dose of azetukalner that is reduced compared to the dose of azetukalner that would be administered to a human not being administered a CYP3A4 inhibitor.

In yet another embodiment, the present disclosure describes azetukalner for use in treating a disease, disorder, or condition in a human in need thereof, wherein the human is being administered a cytochrome P450 3A4 (CYP3A4) inhibitor, the use comprising (a) administering to the human a therapeutically effective amount of azetukalner, and (b) reducing the dose of azetukalner being administered to the human.

In one embodiment, the present disclosure describes azetukalner for use in treating a disease, disorder, or condition in a human in need thereof, the use comprising (a) administering to the human a therapeutically effective amount of azetukalner, (b) subsequently determining that the human is being administered a cytochrome P450 3A4 (CYP3A4) inhibitor, and (c) reducing the dose of azetukalner being administered to the human.

In one embodiment, the present disclosure describes azetukalner for use in treating a disease, disorder, or condition in a human in need thereof, wherein the human is a poor cytochrome P450 3A4 (CYP3A4) metabolizer, the use comprising administering to the human a dose of azetukalner that is reduced compared to the dose of azetukalner that would be administered to a human who is not a poor CYP3A4 metabolizer.

In another embodiment, the present disclosure describes azetukalner for use in treating a disease, disorder, or condition in a human in need thereof, wherein the human is a poor cytochrome P450 3A4 (CYP3A4) metabolizer, the use comprising (a) administering to the human a therapeutically effective amount of azetukalner; and (b) reducing the dose of azetukalner being administered to the human.

In a further embodiment, the present disclosure describes azetukalner for use in treating a disease, disorder, or condition in a human in need thereof, the use comprising (a) administering to the human a therapeutically effective amount of azetukalner, (b) subsequently determining that the human is a poor cytochrome P450 3A4 (CYP3A4) metabolizer, and (c) reducing the dose of azetukalner being administered to the human.

In yet another embodiment, the present disclosure describes azetukalner for use in treating a disease, disorder, or condition in a human in need thereof, the use comprising (a) determining if the human is a poor cytochrome P450 3A4 (CYP3A4) metabolizer, and (b) if the human is a poor CYP3A4 metabolizer, administering to the human a dose of azetukalner that is reduced compared to the dose of azetukalner that would be administered to a human who is not a poor CYP3A4 metabolizer.

In one embodiment, the present disclosure describes azetukalner for use in treating a disease, disorder, or condition in a human in need thereof, wherein the human is being administered a therapeutically effective amount of azetukalner, the use comprising (a) determining if the human is a poor cytochrome P450 3A4 (CYP3A4) metabolizer, and (b) if the human is a poor CYP3A4 metabolizer, reducing the dose of azetukalner being administered to the human.

In one embodiment, the present disclosure describes a use of azetukalner in the manufacture of a medicament for the treatment of a disease, disorder, or condition in a human in need thereof, wherein the human is being administered a cytochrome P450 3A4 (CYP3A4) inhibitor, the treatment comprises (a) administering to the human a therapeutically effective amount of azetukalner, and (b) discontinuing the administration of the CYP3A4 inhibitor.

In another embodiment, the present disclosure describes a use of azetukalner in the manufacture of a medicament treatment of a disease, disorder, or condition in a human in need thereof, wherein the human is being administered a cytochrome P450 3A4 (CYP3A4) inhibitor, the treatment comprises (a) discontinuing the administration of the CYP3A4 inhibitor, and (b) administering to the human a therapeutically effective amount of azetukalner, thereby avoiding the use of the CYP3A4 inhibitor in combination with azetukalner.

In a further embodiment, the present disclosure describes a use of azetukalner in the manufacture of a medicament for the treatment of a disease, disorder, or condition in a human in need thereof, the treatment comprises (a) administering to the human a therapeutically effective amount of azetukalner, (b) subsequently determining that the human is being administered a cytochrome P450 3A4 (CYP3A4) inhibitor, and (c) discontinuing the administration of the CYP3A4 inhibitor.

In yet another embodiment, the present disclosure describes a use of azetukalner in the manufacture of a medicament for the treatment of a disease, disorder, or condition in a human in need thereof, wherein the human is being administered a cytochrome P450 3A4 (CYP3A4) inhibitor, the treatment comprising (a) administering to the human a therapeutically effective amount of azetukalner, and (b) reducing the dose of the CYP3A4 inhibitor being administered to the human.

In one embodiment, the present disclosure describes a use of azetukalner in the manufacture of a medicament for the treatment of a disease, disorder, or condition in a human in need thereof, wherein the human is being administered a cytochrome P450 3A4 (CYP3A4) inhibitor, the treatment comprising (a) reducing the dose of the CYP3A4 inhibitor being administered to the human, and (b) administering to the human a therapeutically effective amount of azetukalner.

In another embodiment, the present disclosure describes a use of azetukalner in the manufacture of a medicament for the treatment of a disease, disorder, or condition in a human in need thereof, the treatment comprising (a) administering to the human a therapeutically effective amount of azetukalner, (b) subsequently determining that the human is being administered a cytochrome P450 3A4 (CYP3A4) inhibitor, and (c) reducing the dosage of the CYP3A4 inhibitor being administered to the human.

In a further embodiment, the present disclosure describes a use of azetukalner in the manufacture of a medicament for the treatment of a disease, disorder, or condition in a human in need thereof, wherein the human is being administered a cytochrome P450 3A4 (CYP3A4) inhibitor, the treatment comprising (a) administering to the human a dose of azetukalner that is reduced compared to the dose of azetukalner that would be administered to a human not being administered a CYP3A4 inhibitor.

In yet another embodiment, the present disclosure describes a use of azetukalner in the manufacture of a medicament for the treatment of a disease, disorder, or condition in a human in need thereof, wherein the human is being administered a cytochrome P450 3A4 (CYP3A4) inhibitor, the treatment comprising (a) administering to the human a therapeutically effective amount of azetukalner, and (b) reducing the dose of azetukalner being administered to the human.

In one embodiment, the present disclosure describes a use of azetukalner in the manufacture of a medicament for the treatment of a disease, disorder, or condition in a human in need thereof, the treatment comprising (a) administering to the human a therapeutically effective amount of azetukalner, (b) subsequently determining that the human is being administered a cytochrome P450 3A4 (CYP3A4) inhibitor, and (c) reducing the dose of azetukalner being administered to the human.

In one embodiment, the present disclosure describes a use of azetukalner in the manufacture of a medicament for the treatment of a disease, disorder, or condition in a human in need thereof, wherein the human is a poor cytochrome P450 3A4 (CYP3A4) metabolizer, the treatment comprising administering to the human a dose of azetukalner that is reduced compared to the dose of azetukalner that would be administered to a human who is not a poor CYP3A4 metabolizer.

In another embodiment, the present disclosure describes a use of azetukalner in the manufacture of a medicament for the treatment of a disease, disorder, or condition in a human in need thereof, wherein the human is a poor cytochrome P450 3A4 (CYP3A4) metabolizer, the treatment comprising (a) administering to the human a therapeutically effective amount of azetukalner; and (b) reducing the dose of azetukalner being administered to the human.

In a further embodiment, the present disclosure describes a use of azetukalner in the manufacture of a medicament for the treatment of a disease, disorder, or condition in a human in need thereof, the treatment comprising (a) administering to the human a therapeutically effective amount of azetukalner, (b) subsequently determining that the human is a poor cytochrome P450 3A4 (CYP3A4) metabolizer, and (c) reducing the dose of azetukalner being administered to the human.

In yet another embodiment, the present disclosure describes a use of azetukalner in the manufacture of a medicament for the treatment of a disease, disorder, or condition in a human in need thereof, the treatment comprising (a) determining if the human is a poor cytochrome P450 3A4 (CYP3A4) metabolizer, and (b) if the human is a poor CYP3A4 metabolizer, administering to the human a dose of azetukalner that is reduced compared to the dose of azetukalner that would be administered to a human who is not a poor CYP3A4 metabolizer.

In one embodiment, the present disclosure describes a use of azetukalner in the manufacture of a medicament for the treatment of a disease, disorder, or condition in a human in need thereof, wherein the human is being administered a therapeutically effective amount of azetukalner, the treatment comprising (a) determining if the human is a poor cytochrome P450 3A4 (CYP3A4) metabolizer, and (b) if the human is a poor CYP3A4 metabolizer, reducing the dose of azetukalner being administered to the human.

In one embodiment, the present disclosure describes a method of treating a disease, disorder, or condition in a human in need thereof, wherein the human is being systemically administered a cytochrome P450 3A4 (CYP3A4) inhibitor, the method comprising (a) orally administering to the human a therapeutically effective amount of azetukalner, and (b) discontinuing the systemic administration of the CYP3A4 inhibitor.

In another embodiment, the present disclosure describes a method of treating a disease, disorder, or condition in a human in need thereof, wherein the human is being systemically administered a cytochrome P450 3A4 (CYP3A4) inhibitor, the method comprising (a) discontinuing the systemic administration of the CYP3A4 inhibitor, and (b) orally administering to the human a therapeutically effective amount of azetukalner, thereby avoiding the use of the CYP3A4 inhibitor in combination with azetukalner.

In a further embodiment, the present disclosure describes a method of treating a disease, disorder, or condition in a human in need thereof, the method comprising (a) orally administering to the human a therapeutically effective amount of azetukalner, (b) subsequently determining that the human is being systemically administered a cytochrome P450 3A4 (CYP3A4) inhibitor, and (c) discontinuing the systemic administration of the CYP3A4 inhibitor.

In one embodiment, the present disclosure describes an improvement in a method of treating a disease, disorder, or condition in a human in need thereof, the method comprising orally administering to the human a therapeutically effective amount of azetukalner, wherein the human is being systemically administered a cytochrome P450 3A4 (CYP3A4) inhibitor, the improvement comprising discontinuing the systemic administration of the CYP3A4 inhibitor.

In another embodiment, the present disclosure describes an improvement in a method of treating a disease, disorder, or condition in a human in need thereof, wherein the human is being systemically administered a cytochrome P450 3A4 (CYP3A4) inhibitor, the improvement comprising (a) discontinuing the systemic administration of the CYP3A4 inhibitor, and (b) orally administering to the human a therapeutically effective amount of azetukalner, thereby avoiding the use of the CYP3A4 inhibitor in combination with azetukalner.

In a further embodiment, the present disclosure describes an improvement in a method of treating a disease, disorder, or condition in a human in need thereof, the improvement comprising (a) orally administering to the human a therapeutically effective amount of azetukalner, (b) subsequently determining that the human is being systemically administered a cytochrome P450 3A4 (CYP3A4) inhibitor, and (c) discontinuing the systemic administration of the CYP3A4 inhibitor.

In yet another embodiment, the present disclosure describes a method of treating a disease, disorder, or condition in a human in need thereof, wherein the human is being systemically administered a cytochrome P450 3A4 (CYP3A4) inhibitor, the method comprising (a) orally administering to the human a therapeutically effective amount of azetukalner, and (b) reducing the dose of the CYP3A4 inhibitor being systemically administered to the human.

In one embodiment, the present disclosure describes a method of treating a disease, disorder, or condition in a human in need thereof, wherein the human is being systemically administered a cytochrome P450 3A4 (CYP3A4) inhibitor, the method comprising (a) reducing the dose of the 14516914 21U61 CYP3A4 inhibitor being systemically administered to the human, and (b) orally administering to the human a therapeutically effective amount of azetukalner.

In another embodiment, the present disclosure describes a method of treating a disease, disorder, or condition in a human in need thereof, the method comprising (a) orally administering to the human a therapeutically effective amount of azetukalner, (b) subsequently determining that the human is being systemically administered a cytochrome P450 3A4 (CYP3A4) inhibitor, and (c) reducing the dosage of the CYP3A4 inhibitor being systemically administered to the human.

In a further embodiment, the present disclosure describes an improvement in a method of treating a disease, disorder, or condition in a human in need thereof, the method comprising orally administering to the human a therapeutically effective amount of azetukalner, wherein the human is being systemically administered a cytochrome P450 3A4 (CYP3A4) inhibitor, the improvement comprising reducing the dose of the CYP3A4 inhibitor being systemically administered to the human.

In yet another embodiment, the present disclosure describes an improvement in a method of treating a disease, disorder, or condition in a human in need thereof, wherein the human is being systemically administered a cytochrome P450 3A4 (CYP3A4) inhibitor, the improvement comprising (a) orally administering to the human a therapeutically effective amount of azetukalner, and (b) reducing the dose of the CYP3A4 inhibitor being systemically administered to the human.

In one embodiment, the present disclosure describes an improvement in a method of treating a disease, disorder, or condition in a human in need thereof, wherein the human is being systemically administered a cytochrome P450 3A4 (CYP3A4) inhibitor, the improvement comprising, (a) reducing the dose of the CYP3A4 inhibitor being systemically administered to the human, and (b) orally administering to the human a therapeutically effective amount of azetukalner.

In another embodiment, the present disclosure describes an improvement in a method of treating a disease, disorder, or condition in a human in need thereof, the improvement comprising (a) orally administering to the human a therapeutically effective amount of azetukalner, (b) subsequently determining that the human is being systemically administered a cytochrome P450 3A4 (CYP3A4) inhibitor, and (c) reducing the dose of the CYP3A4 inhibitor being systemically administered to the human.

In a further embodiment, the present disclosure describes a method of treating a disease, disorder, or condition in a human in need thereof, wherein the human is being systemically administered a cytochrome P450 3A4 (CYP3A4) inhibitor, the method comprising (a) orally administering to the human a dose of azetukalner that is reduced compared to the dose of azetukalner that would be administered to a human not being systemically administered a CYP3A4 inhibitor.

In yet another embodiment, the present disclosure describes a method of treating a disease, disorder, or condition in a human in need thereof, wherein the human is being systemically administered a cytochrome P450 3A4 (CYP3A4) inhibitor, the method comprising (a) orally administering to the human a therapeutically effective amount of azetukalner, and (b) reducing the dose of azetukalner being administered to the human.

In one embodiment, the present disclosure describes a method of treating a disease, disorder, or condition in a human in need thereof, the method comprising (a) orally administering to the human a therapeutically effective amount of azetukalner, (b) subsequently determining that the human is being systemically administered a cytochrome P450 3A4 (CYP3A4) inhibitor, and (c) reducing the dose of azetukalner being administered to the human.

In another embodiment, the present disclosure describes an improvement in a method of treating a disease, disorder, or condition in a human in need thereof, the method comprising orally administering to the human a therapeutically effective amount of azetukalner, wherein the human is being systemically administered a cytochrome P450 3A4 (CYP3A4) inhibitor, the improvement comprising reducing the dose of azetukalner being administered to the human.

In a further embodiment, the present disclosure describes an improvement in a method of treating a disease, disorder, or condition in a human in need thereof, the method comprising orally administering to the human a therapeutically effective amount of azetukalner, wherein the human is being systemically administered a cytochrome P450 3A4 (CYP3A4) inhibitor, the improvement comprising orally administering to the human a dose of azetukalner that is reduced compared to the dose of azetukalner that would be administered to a human not being systemically administered a CYP3A4 inhibitor.

In yet another embodiment, the present disclosure describes an improvement in a method of treating a disease, disorder, or condition in a human in need thereof, the method comprising orally administering to the human a therapeutically effective amount of azetukalner, wherein the human is being systemically administered a cytochrome P450 3A4 (CYP3A4) inhibitor, the improvement comprising (a) orally administering to the human a therapeutically effective amount of azetukalner, (b) subsequently determining that the human is being systemically administered a cytochrome P450 3A4 (CYP3A4) inhibitor, and (c) reducing the dose of azetukalner being administered to the human.

In one embodiment, the present disclosure describes a method of treating a disease, disorder, or condition in a human in need thereof, wherein the human is a poor cytochrome P450 3A4 (CYP3A4) metabolizer, the method comprising orally administering to the human a dose of azetukalner that is reduced compared to the dose of azetukalner that would be administered to a human who is not a poor CYP3A4 metabolizer.

In another embodiment, the present disclosure describes a method of treating a disease, disorder, or condition in a human in need thereof, wherein the human is a poor cytochrome P450 3A4 (CYP3A4) metabolizer, the method comprising (a) orally administering to the human a therapeutically effective amount of azetukalner; and (b) reducing the dose of azetukalner being administered to the human.

In a further embodiment, the present disclosure describes a method of treating a disease, disorder, or condition in a human in need thereof, the method comprising (a) orally administering to the human a therapeutically effective amount of azetukalner, (b) subsequently determining that the human is a poor cytochrome P450 3A4 (CYP3A4) metabolizer, and (c) reducing the dose of azetukalner being administered to the human.

In yet another embodiment, the present disclosure describes a method of treating a disease, disorder, or condition in a human in need thereof, the method comprising (a) determining if the human is a poor cytochrome P450 3A4 (CYP3A4) metabolizer, and (b) if the human is a poor CYP3A4 metabolizer, orally administering to the human a dose of azetukalner that is reduced compared to the dose of azetukalner that would be administered to a human who is not a poor CYP3A4 metabolizer.

In one embodiment, the present disclosure describes method of treating a disease, disorder, or condition in a human in need thereof, wherein the human is being orally administered a therapeutically effective amount of azetukalner, the method comprising (a) determining if the human is a poor cytochrome P450 3A4 (CYP3A4) metabolizer, and (b) if the human is a poor CYP3A4 metabolizer, reducing the dose of azetukalner being administered to the human.

In another embodiment, the present disclosure describes an improvement in a method of treating a disease, disorder, or condition in a human in need thereof, the method comprising orally administering to the human a therapeutically effective amount of azetukalner, wherein the human is a poor cytochrome P450 3A4 (CYP3A4) metabolizer, the improvement comprising orally administering to the human a dose of azetukalner that is reduced compared to the dose of azetukalner that would be administered to a human who is not a poor CYP3A4 metabolizer.

In a further embodiment, the present disclosure describes an improvement in a method of treating a disease, disorder, or condition in a human in need thereof, the method comprising orally administering to the human a therapeutically effective amount of azetukalner, wherein the human is a poor cytochrome P450 3A4 (CYP3A4) metabolizer, the improvement comprising reducing the dose of azetukalner being administered to the human.

In yet another embodiment, the present disclosure describes an improvement in a method of treating a disease, disorder, or condition in a human in need thereof, the improvement comprising (a) orally administering to the human a therapeutically effective amount of azetukalner, (b) subsequently determining that the human is a poor cytochrome P450 3A4 (CYP3A4) metabolizer, and (c) reducing the dose of azetukalner being administered to the human.

In one embodiment, the present disclosure describes an improvement in a method of treating a disease, disorder, or condition in a human in need thereof, the improvement comprising (a) determining if the human is a poor cytochrome P450 3A4 (CYP3A4) metabolizer, and (b) if the human is a poor CYP3A4 metabolizer, orally administering to the human a dose of azetukalner that is reduced compared to the dose of azetukalner that would be administered to a human who is not a poor CYP3A4 metabolizer.

In another embodiment, the present disclosure describes an improvement in a method of treating a disease, disorder, or condition in a human in need thereof, wherein the human is being orally administered a therapeutically effective amount of azetukalner, the improvement comprising (a) determining if the human is a poor cytochrome P450 3A4 (CYP3A4) metabolizer, and (b) if the human is a poor CYP3A4 metabolizer, reducing the dose of azetukalner being administered to the human.

In one embodiment, the present disclosure describes a use of azetukalner in treating a disease, disorder, or condition in a human in need thereof, wherein the human is being systemically administered a cytochrome P450 3A4 (CYP3A4) inhibitor, the use comprising (a) orally administering to the human a therapeutically effective amount of azetukalner, and (b) discontinuing the systemic administration of the CYP3A4 inhibitor.

In another embodiment, the present disclosure describes a use of azetukalner in treating a disease, disorder, or condition in a human in need thereof, wherein the human is being systemically administered a cytochrome P450 3A4 (CYP3A4) inhibitor, the use comprising (a) discontinuing the systemic administration of the CYP3A4 inhibitor, and (b) orally administering to the human a therapeutically effective amount of azetukalner, thereby avoiding the use of the CYP3A4 inhibitor in combination with azetukalner.

In a further embodiment, the present disclosure describes a use of azetukalner in treating a disease, disorder, or condition in a human in need thereof, the use comprising (a) orally administering to the human a therapeutically effective amount of azetukalner, (b) subsequently determining that the human is being systemically administered a cytochrome P450 3A4 (CYP3A4) inhibitor, and (c) discontinuing the systemic administration of the CYP3A4 inhibitor.

In yet another embodiment, the present disclosure describes a use of azetukalner in treating a disease, disorder, or condition in a human in need thereof, wherein the human is being systemically administered a cytochrome P450 3A4 (CYP3A4) inhibitor, the use comprising (a) orally administering to the human a therapeutically effective amount of azetukalner, and (b) reducing the dose of the CYP3A4 inhibitor being systemically administered to the human.

In one embodiment, the present disclosure describes a use of azetukalner in treating a disease, disorder, or condition in a human in need thereof, wherein the human is being systemically administered a cytochrome P450 3A4 (CYP3A4) inhibitor, the use comprising (a) reducing the dose of the CYP3A4 inhibitor being systemically administered to the human, and (b) orally administering to the human a therapeutically effective amount of azetukalner.

In another embodiment, the present disclosure describes a use of azetukalner in treating a disease, disorder, or condition in a human in need thereof, the use comprising (a) orally administering to the human a therapeutically effective amount of azetukalner, (b) subsequently determining that the human is being systemically administered a cytochrome P450 3A4 (CYP3A4) inhibitor, and (c) reducing the dosage of the CYP3A4 inhibitor being systemically administered to the human.

In a further embodiment, the present disclosure describes a use of azetukalner in treating a disease, disorder, or condition in a human in need thereof, wherein the human is being systemically administered a cytochrome P450 3A4 (CYP3A4) inhibitor, the use comprising (a) orally administering to the human a dose of azetukalner that is reduced compared to the dose of azetukalner that would be administered to a human not being systemically administered a CYP3A4 inhibitor.

In yet another embodiment, the present disclosure describes a use of azetukalner in treating a disease, disorder, or condition in a human in need thereof, wherein the human is being systemically administered a cytochrome P450 3A4 (CYP3A4) inhibitor, the use comprising (a) orally administering to the human a therapeutically effective amount of azetukalner, and (b) reducing the dose of azetukalner being administered to the human.

In one embodiment, the present disclosure describes a use of azetukalner in treating a disease, disorder, or condition in a human in need thereof, the use comprising (a) orally administering to the human a therapeutically effective amount of azetukalner, (b) subsequently determining that the human is being systemically administered a cytochrome P450 3A4 (CYP3A4) inhibitor, and (c) reducing the dose of azetukalner being administered to the human.

In one embodiment, the present disclosure describes a use of azetukalner in treating a disease, disorder, or condition in a human in need thereof, wherein the human is a poor cytochrome P450 3A4 (CYP3A4) metabolizer, the use comprising orally administering to the human a dose of azetukalner that is reduced compared to the dose of azetukalner that would be administered to a human who is not a poor CYP3A4 metabolizer.

In another embodiment, the present disclosure describes a use of azetukalner in treating a disease, disorder, or condition in a human in need thereof, wherein the human is a poor cytochrome P450 3A4 (CYP3A4) metabolizer, the use comprising (a) orally administering to the human a therapeutically effective amount of azetukalner; and (b) reducing the dose of azetukalner being administered to the human.

In a further embodiment, the present disclosure describes a use of azetukalner in treating a disease, disorder, or condition in a human in need thereof, the use comprising (a) orally administering to the human a therapeutically effective amount of azetukalner, (b) subsequently determining that the human is a poor cytochrome P450 3A4 (CYP3A4) metabolizer, and (c) reducing the dose of azetukalner being administered to the human.

In yet another embodiment, the present disclosure describes a use of azetukalner in treating a disease, disorder, or condition in a human in need thereof, the use comprising (a) determining if the human is a poor cytochrome P450 3A4 (CYP3A4) metabolizer, and (b) if the human is a poor CYP3A4 metabolizer, orally administering to the human a dose of azetukalner that is reduced compared to the dose of azetukalner that would be administered to a human who is not a poor CYP3A4 metabolizer.

In one embodiment, the present disclosure describes a use of azetukalner in treating a disease, disorder, or condition in a human in need thereof, wherein the human is being orally administered a therapeutically effective amount of azetukalner, the use comprising (a) determining if the human is a poor cytochrome P450 3A4 (CYP3A4) metabolizer, and (b) if the human is a poor CYP3A4 metabolizer, reducing the dose of azetukalner being administered to the human.

In one embodiment, the present disclosure describes azetukalner for use in treating a disease, disorder, or condition in a human in need thereof, wherein the human is being systemically administered a cytochrome P450 3A4 (CYP3A4) inhibitor, the use comprising (a) orally administering to the human a therapeutically effective amount of azetukalner, and (b) discontinuing the systemic administration of the CYP3A4 inhibitor.

In another embodiment, the present disclosure describes azetukalner for use in treating a disease, disorder, or condition in a human in need thereof, wherein the human is being systemically administered a cytochrome P450 3A4 (CYP3A4) inhibitor, the use comprising (a) discontinuing the systemic administration of the CYP3A4 inhibitor, and (b) orally administering to the human a therapeutically effective amount of azetukalner, thereby avoiding the use of the CYP3A4 inhibitor in combination with azetukalner.

In a further embodiment, the present disclosure describes azetukalner for use in treating a disease, disorder, or condition in a human in need thereof, the use comprising (a) orally administering to the human a therapeutically effective amount of azetukalner, (b) subsequently determining that the human is being systemically administered a cytochrome P450 3A4 (CYP3A4) inhibitor, and (c) discontinuing the systemic administration of the CYP3A4 inhibitor.

In yet another embodiment, the present disclosure describes azetukalner for use in treating a disease, disorder, or condition in a human in need thereof, wherein the human is being systemically administered a cytochrome P450 3A4 (CYP3A4) inhibitor, the use comprising (a) orally administering to the human a therapeutically effective amount of azetukalner, and (b) reducing the dose of the CYP3A4 inhibitor being systemically administered to the human.

In one embodiment, the present disclosure describes azetukalner for use in treating a disease, disorder, or condition in a human in need thereof, wherein the human is being systemically administered a cytochrome P450 3A4 (CYP3A4) inhibitor, the use comprising (a) reducing the dose of the CYP3A4 inhibitor being systemically administered to the human, and (b) orally administering to the human a therapeutically effective amount of azetukalner.

In another embodiment, the present disclosure describes azetukalner for use in treating a disease, disorder, or condition in a human in need thereof, the use comprising (a) orally administering to the human a therapeutically effective amount of azetukalner, (b) subsequently determining that the human is being systemically administered a cytochrome P450 3A4 (CYP3A4) inhibitor, and (c) reducing the dosage of the CYP3A4 inhibitor being systemically administered to the human.

In a further embodiment, the present disclosure describes azetukalner for use in treating a disease, disorder, or condition in a human in need thereof, wherein the human is being systemically administered a cytochrome P450 3A4 (CYP3A4) inhibitor, the use comprising (a) orally administering to the human a dose of azetukalner that is reduced compared to the dose of azetukalner that would be administered to a human not being systemically administered a CYP3A4 inhibitor.

In yet another embodiment, the present disclosure describes azetukalner for use in treating a disease, disorder, or condition in a human in need thereof, wherein the human is being systemically administered a cytochrome P450 3A4 (CYP3A4) inhibitor, the use comprising (a) orally administering to the human a therapeutically effective amount of azetukalner, and (b) reducing the dose of azetukalner being administered to the human.

In one embodiment, the present disclosure describes azetukalner for use in treating a disease, disorder, or condition in a human in need thereof, the use comprising (a) orally administering to the human a therapeutically effective amount of azetukalner, (b) subsequently determining that the human is being systemically administered a cytochrome P450 3A4 (CYP3A4) inhibitor, and (c) reducing the dose of azetukalner being administered to the human.

In one embodiment, the present disclosure describes azetukalner for use in treating a disease, disorder, or condition in a human in need thereof, wherein the human is a poor cytochrome P450 3A4 (CYP3A4) metabolizer, the use comprising orally administering to the human a dose of azetukalner that is reduced compared to the dose of azetukalner that would be administered to a human who is not a poor CYP3A4 metabolizer.

In another embodiment, the present disclosure describes azetukalner for use in treating a disease, disorder, or condition in a human in need thereof, wherein the human is a poor cytochrome P450 3A4 (CYP3A4) metabolizer, the use comprising (a) orally administering to the human a therapeutically effective amount of azetukalner; and (b) reducing the dose of azetukalner being administered to the human.

In a further embodiment, the present disclosure describes azetukalner for use in treating a disease, disorder, or condition in a human in need thereof, the use comprising (a) orally administering to the human a therapeutically effective amount of azetukalner, (b) subsequently determining that the human is a poor cytochrome P450 3A4 (CYP3A4) metabolizer, and (c) reducing the dose of azetukalner being administered to the human.

In yet another embodiment, the present disclosure describes azetukalner for use in treating a disease, disorder, or condition in a human in need thereof, the use comprising (a) determining if the human is a poor cytochrome P450 3A4 (CYP3A4) metabolizer, and (b) if the human is a poor CYP3A4 metabolizer, orally administering to the human a dose of azetukalner that is reduced compared to the dose of azetukalner that would be administered to a human who is not a poor CYP3A4 metabolizer.

In one embodiment, the present disclosure describes azetukalner for use in treating a disease, disorder, or condition in a human in need thereof, wherein the human is being orally administered a therapeutically effective amount of azetukalner, the use comprising (a) determining if the human is a poor cytochrome P450 3A4 (CYP3A4) metabolizer, and (b) if the human is a poor CYP3A4 metabolizer, reducing the dose of azetukalner being administered to the human.

In one embodiment, the present disclosure describes a use of azetukalner in the manufacture of a medicament for the treatment of a disease, disorder, or condition in a human in need thereof, wherein the human is being systemically administered a cytochrome P450 3A4 (CYP3A4) inhibitor, the treatment comprises (a) orally administering to the human a therapeutically effective amount of azetukalner, and (b) discontinuing the systemic administration of the CYP3A4 inhibitor.

In another embodiment, the present disclosure describes a use of azetukalner in the manufacture of a medicament treatment of a disease, disorder, or condition in a human in need thereof, wherein the human is being systemically administered a cytochrome P450 3A4 (CYP3A4) inhibitor, the treatment comprises (a) discontinuing the systemic administration of the CYP3A4 inhibitor, and (b) orally administering to the human a therapeutically effective amount of azetukalner, thereby avoiding the use of the CYP3A4 inhibitor in combination with azetukalner.

In a further embodiment, the present disclosure describes a use of azetukalner in the manufacture of a medicament for the treatment of a disease, disorder, or condition in a human in need thereof, the treatment comprises (a) orally administering to the human a therapeutically effective amount of azetukalner, (b) subsequently determining that the human is being systemically administered a cytochrome P450 3A4 (CYP3A4) inhibitor, and (c) discontinuing the systemic administration of the CYP3A4 inhibitor.

In yet another embodiment, the present disclosure describes a use of azetukalner in the manufacture of a medicament for the treatment of a disease, disorder, or condition in a human in need thereof, wherein the human is being systemically administered a cytochrome P450 3A4 (CYP3A4) inhibitor, the treatment comprising (a) orally administering to the human a therapeutically effective amount of azetukalner, and (b) reducing the dose of the CYP3A4 inhibitor being systemically administered to the human.

In one embodiment, the present disclosure describes a use of azetukalner in the manufacture of a medicament for the treatment of a disease, disorder, or condition in a human in need thereof, wherein the human is being systemically administered a cytochrome P450 3A4 (CYP3A4) inhibitor, the treatment comprising (a) reducing the dose of the CYP3A4 inhibitor being systemically administered to the human, and (b) orally administering to the human a therapeutically effective amount of azetukalner.

In another embodiment, the present disclosure describes a use of azetukalner in the manufacture of a medicament for the treatment of a disease, disorder, or condition in a human in need thereof, the treatment comprising (a) orally administering to the human a therapeutically effective amount of azetukalner, (b) subsequently determining that the human is being systemically administered a cytochrome P450 3A4 (CYP3A4) inhibitor, and (c) reducing the dosage of the CYP3A4 inhibitor being systemically administered to the human.

In a further embodiment, the present disclosure describes a use of azetukalner in the manufacture of a medicament for the treatment of a disease, disorder, or condition in a human in need thereof, wherein the human is being systemically administered a cytochrome P450 3A4 (CYP3A4) inhibitor, the treatment comprising (a) orally administering to the human a dose of azetukalner that is reduced compared to the dose of azetukalner that would be administered to a human not being systemically administered a CYP3A4 inhibitor.

In yet another embodiment, the present disclosure describes a use of azetukalner in the manufacture of a medicament for the treatment of a disease, disorder, or condition in a human in need thereof, wherein the human is being systemically administered a cytochrome P450 3A4 (CYP3A4) inhibitor, the treatment comprising (a) orally administering to the human a therapeutically effective amount of azetukalner, and (b) reducing the dose of azetukalner being administered to the human.

In one embodiment, the present disclosure describes a use of azetukalner in the manufacture of a medicament for the treatment of a disease, disorder, or condition in a human in need thereof, the treatment comprising (a) orally administering to the human a therapeutically effective amount of azetukalner, (b) subsequently determining that the human is being systemically administered a cytochrome P450 3A4 (CYP3A4) inhibitor, and (c) reducing the dose of azetukalner being administered to the human.

In one embodiment, the present disclosure describes a use of azetukalner in the manufacture of a medicament for the treatment of a disease, disorder, or condition in a human in need thereof, wherein the human is a poor cytochrome P450 3A4 (CYP3A4) metabolizer, the treatment comprising orally administering to the human a dose of azetukalner that is reduced compared to the dose of azetukalner that would be administered to a human who is not a poor CYP3A4 metabolizer.

In another embodiment, the present disclosure describes a use of azetukalner in the manufacture of a medicament for the treatment of a disease, disorder, or condition in a human in need thereof, wherein the human is a poor cytochrome P450 3A4 (CYP3A4) metabolizer, the treatment comprising (a) orally administering to the human a therapeutically effective amount of azetukalner; and (b) reducing the dose of azetukalner being administered to the human.

In a further embodiment, the present disclosure describes a use of azetukalner in the manufacture of a medicament for the treatment of a disease, disorder, or condition in a human in need thereof, the treatment comprising (a) orally administering to the human a therapeutically effective amount of azetukalner, (b) subsequently determining that the human is a poor cytochrome P450 3A4 (CYP3A4) metabolizer, and (c) reducing the dose of azetukalner being administered to the human.

In yet another embodiment, the present disclosure describes a use of azetukalner in the manufacture of a medicament for the treatment of a disease, disorder, or condition in a human in need thereof, the treatment comprising (a) determining if the human is a poor cytochrome P450 3A4 (CYP3A4) metabolizer, and (b) if the human is a poor CYP3A4 metabolizer, orally administering to the human a dose of azetukalner that is reduced compared to the dose of azetukalner that would be administered to a human who is not a poor CYP3A4 metabolizer.

In one embodiment, the present disclosure describes a use of azetukalner in the manufacture of a medicament for the treatment of a disease, disorder, or condition in a human in need thereof, wherein the human is being orally administered a therapeutically effective amount of azetukalner, the treatment comprising (a) determining if the human is a poor cytochrome P450 3A4 (CYP3A4) metabolizer, and (b) if the human is a poor CYP3A4 metabolizer, reducing the dose of azetukalner being administered to the human.

In certain embodiments, azetukalner the CYP3A4 inhibitor is a strong CYP3A4 inhibitor. In certain embodiments, the strong CYP3A4 inhibitor is selected from a group consisting of atazanavir, adagrasib, ceritinib, clarithromycin, cobicistat, darunavir, delavirdine, idelalisib, itraconazole, ketoconazole, nefazodone, nelfinavir, posaconazole, ribociclib, ritonavir, telithromycin, tucatinib, voriconazole, or a combination thereof. In certain embodiments, the strong CYP3A4 inhibitor is a combination product comprising ritonavir. For example, the strong CYP3A4 inhibitor may be a combination of (a) nirmatrelvir and ritonavir, (b) elvitegravir and ritonavir, (c) lopinavir and ritonavir, (d) paritaprevir and ritonavir, (e) saquinavir and ritonavir, or (f) tipranavir and ritonavir. In certain embodiments, the strong CYP3A4 inhibitor comprises a combination of nirmatrelvir and ritonavir.

In certain embodiments, the CYP3A4 inhibitor is a moderate CYP3A4 inhibitor. In certain embodiments, the moderate CYP3A4 inhibitor is selected from a group consisting of amiodarone. aprepitant, ciprofloxacin, conivaptan, crizotinib, diltiazem, erythromycin, fluconazole, imatinib, isavuconazole, miconazole, diltiazem, verapamil, delavirdine, amprenavir, fosamprenavir, conivaptan, or a combination thereof.

In certain embodiments, azetukalner is administered to a subject in need thereof with a CYP3A4 inhibitor for an initial co-administration period. In certain embodiments, the initial co-administration period is no more than ten days, no more than nine days, no more than eight days, no more than seven days, no more than six days, no more than five days, no more than four days, no more than three days, no more than two days, or no more than one day. In certain embodiments, the initial co-administration period is no more than fourteen days. In certain embodiments, the initial co-administration period is no more than ten days. In certain embodiments, the initial co-administration period is no more than nine days. In certain embodiments, the initial co-administration period is no more than eight days. In certain embodiments, the initial co-administration period is no more than seven days. In certain embodiments, the initial co-administration period is no more than six days. In certain embodiments, the initial co-administration period is no more than five days. In certain embodiments, the initial co-administration period is no more than four days. In certain embodiments, the initial co-administration period is no more than three days. In certain embodiments, the initial co-administration period is no more than two days. In certain embodiments, the initial co-administration period is no more than one day.

In certain embodiments the initial co-administration period is between one and ten days, between two and nine days, between three and eight days, between four and seven days, between five and six days, between six and eight days, or between five and nine days. In certain embodiments, the initial co-administration period is between one and fourteen days. In certain embodiments, the initial co-administration period is between four and fourteen days. In certain embodiments, the initial co-administration period is between one and ten days. In certain embodiments, the initial co-administration period is between two and nine days. In certain embodiments, the initial co-administration period is between three and eight days. In certain embodiments, the initial co-administration period is between four and seven days. In certain embodiments, the initial co-administration period is between five and six days. In certain embodiments, the initial co-administration period is between six and eight days. In certain embodiments, the initial co-administration period is between five and nine days.

In certain embodiments, the initial co-administration period is fourteen days. In certain embodiments, the initial co-administration period is seven days.

In some embodiments, the present disclosure provides a method of treating a disease, disorder, or condition in a human in need thereof, wherein the human is being systemically administered a cytochrome P450 3A4 (CYP3A4) inhibitor, the method comprising: a) orally administering to the human a therapeutically effective amount of azetukalner; and b) discontinuing the systemic administration of the CYP3A4 inhibitor after an initial co-administration period of no more than fourteen days.

In some embodiments, the present disclosure provides a method of treating a disease, disorder, or condition in a human in need thereof, wherein the human is being systemically administered a cytochrome P450 3A4 (CYP3A4) inhibitor, the method comprising: a) orally administering to the human a therapeutically effective amount of azetukalner; and b) discontinuing the systemic administration of the CYP3A4 inhibitor after an initial co-administration period of no more than seven days.

In some embodiments, the present disclosure provides a method of treating a disease, disorder, or condition in a human in need thereof, the method comprising: a) orally administering to the human a therapeutically effective amount of azetukalner; b) subsequently determining that the human is being systemically administered a cytochrome P450 3A4 (CYP3A4) inhibitor; and c) discontinuing the systemic administration of the CYP3A4 inhibitor after an initial co-administration period of no more than fourteen days.

In some embodiments, the present disclosure provides a method of treating a disease, disorder, or condition in a human in need thereof, the method comprising: a) orally administering to the human a therapeutically effective amount of azetukalner; b) subsequently determining that the human is being systemically administered a cytochrome P450 3A4 (CYP3A4) inhibitor; and c) discontinuing the systemic administration of the CYP3A4 inhibitor after an initial co-administration period of no more than seven days.

In some embodiments, the present disclosure provides a method of treating a disease, disorder, or condition in a human in need thereof, wherein the human is being systemically administered a cytochrome P450 3A4 (CYP3A4) inhibitor, the method comprising: a) orally administering to the human a therapeutically effective amount of azetukalner; and b) reducing the dose of the CYP3A4 inhibitor being systemically administered to the human after an initial co-administration period of fourteen days with azetukalner.

In some embodiments, the present disclosure provides a method of treating a disease, disorder, or condition in a human in need thereof, wherein the human is being systemically administered a cytochrome P450 3A4 (CYP3A4) inhibitor, the method comprising: a) orally administering to the human a therapeutically effective amount of azetukalner; and b) reducing the dose of the CYP3A4 inhibitor being systemically administered to the human after an initial co-administration period of seven days with azetukalner.

In some embodiments, the present disclosure provides a method of treating a disease, disorder, or condition in a human in need thereof, the method comprising: a) orally administering to the human a therapeutically effective amount of azetukalner; b) subsequently determining that the human is being systemically administered a cytochrome P450 3A4 (CYP3A4) inhibitor; and c) reducing the dosage of the CYP3A4 inhibitor being systemically administered to the human after an initial co-administration period of fourteen days with azetukalner.

In some embodiments, the present disclosure provides a method of treating a disease, disorder, or condition in a human in need thereof, the method comprising: a) orally administering to the human a therapeutically effective amount of azetukalner; b) subsequently determining that the human is being systemically administered a cytochrome P450 3A4 (CYP3A4) inhibitor; and c) reducing the dosage of the CYP3A4 inhibitor being systemically administered to the human after an initial co-administration period of seven days with azetukalner.

In some embodiments, the present disclosure provides a method of treating a disease, disorder, or condition in a human in need thereof, wherein the human is being systemically administered a cytochrome P450 3A4 (CYP3A4) inhibitor, the method comprising: a) discontinuing the systemic administration of the CYP3A4 inhibitor after an initial co-administration period of no more than fourteen days with azetukalner; and b) orally administering to the human a therapeutically effective amount of azetukalner, thereby avoiding the use of the CYP3A4 inhibitor in combination with azetukalner.

In some embodiments, the present disclosure provides a method of treating a disease, disorder, or condition in a human in need thereof, wherein the human is being systemically administered a cytochrome P450 3A4 (CYP3A4) inhibitor, the method comprising: a) discontinuing the systemic administration of the CYP3A4 inhibitor after an initial co-administration period of no more than seven days with azetukalner; and b) orally administering to the human a therapeutically effective amount of azetukalner, thereby avoiding the use of the CYP3A4 inhibitor in combination with azetukalner.

In some embodiments, the present disclosure provides a method of treating a disease, disorder, or condition in a human in need thereof, wherein the human is being systemically administered a cytochrome P450 3A4 (CYP3A4) inhibitor, the method comprising: a) discontinuing the systemic administration of the CYP3A4 inhibitor after an initial concomitant systemic treatment period of no more than fourteen days with azetukalner; and b) orally administering to the human a therapeutically effective amount of azetukalner, thereby avoiding the concomitant systemic treatment with the CYP3A4 inhibitor in combination with azetukalner.

In some embodiments, the present disclosure provides a method of treating a disease, disorder, or condition in a human in need thereof, wherein the human is being systemically administered a cytochrome P450 3A4 (CYP3A4) inhibitor, the method comprising: a) discontinuing the systemic administration of the CYP3A4 inhibitor after an initial concomitant systemic treatment period of no more than seven days with azetukalner; and b) orally administering to the human a therapeutically effective amount of azetukalner, thereby avoiding the concomitant systemic treatment with the CYP3A4 inhibitor in combination with azetukalner.

In some embodiments, the present disclosure provides a method of treating a disease, disorder, or condition in a human in need thereof, wherein the human is being systemically administered a cytochrome P450 3A4 (CYP3A4) inhibitor, the method comprising: a) reducing the dose of the CYP3A4 inhibitor being systemically administered to the human after an initial co-administration period of no more than fourteen days with azetukalner; and b) orally administering to the human a therapeutically effective amount of azetukalner.

In some embodiments, the present disclosure provides a method of treating a disease, disorder, or condition in a human in need thereof, wherein the human is being systemically administered a cytochrome P450 3A4 (CYP3A4) inhibitor, the method comprising: a) reducing the dose of the CYP3A4 inhibitor being systemically administered to the human after an initial co-administration period of no more than seven days with azetukalner; and b) orally administering to the human a therapeutically effective amount of azetukalner.

In some embodiments, the present disclosure provides a method of treating a disease, disorder, or condition in a human in need thereof, wherein the human is being systemically administered a strong cytochrome P450 3A4 (CYP3A4) inhibitor, the method comprising: a) discontinuing the systemic administration of the strong CYP3A4 inhibitor after an initial co-administration period of no more than fourteen days with azetukalner; and b) orally administering to the human a therapeutically effective amount of azetukalner, thereby avoiding the use of the strong CYP3A4 inhibitor in combination with azetukalner. In some embodiments, the strong CYP34A inhibitor is itraconazole.

In some embodiments, the present disclosure provides a method of treating a disease, disorder, or condition in a human in need thereof, wherein the human is being systemically administered a strong cytochrome P450 3A4 (CYP3A4) inhibitor, the method comprising: a) discontinuing the systemic administration of the strong CYP3A4 inhibitor after an initial co-administration period of no more than seven days with azetukalner; and b) orally administering to the human a therapeutically effective amount of azetukalner, thereby avoiding the use of the strong CYP3A4 inhibitor in combination with azetukalner. In some embodiments, the strong CYP34A inhibitor is itraconazole.

In some embodiments, the present disclosure provides a method of treating a disease, disorder, or condition in a human in need thereof, wherein the human is being systemically administered a strong cytochrome P450 3A4 (CYP3A4) inhibitor, the method comprising: a) reducing the dose of the strong CYP3A4 inhibitor being systemically administered to the human after an initial co-administration period of no more than fourteen days with azetukalner; and b) orally administering to the human a therapeutically effective amount of azetukalner. In some embodiments, the strong CYP34A inhibitor is itraconazole.

In some embodiments, the present disclosure provides a method of treating a disease, disorder, or condition in a human in need thereof, wherein the human is being systemically administered a strong cytochrome P450 3A4 (CYP3A4) inhibitor, the method comprising: a) reducing the dose of the strong CYP3A4 inhibitor being systemically administered to the human after an initial co-administration period of no more than seven days with azetukalner; and b) orally administering to the human a therapeutically effective amount of azetukalner. In some embodiments, the strong CYP34A inhibitor is itraconazole.

In some embodiments, the present disclosure provides a method of treating a disease, disorder, or condition in a human in need thereof, wherein the human is being systemically administered a strong cytochrome P450 3A4 (CYP3A4) inhibitor, the method comprising: a) orally administering to the human a therapeutically effective amount of azetukalner; and b) discontinuing the systemic administration of the strong CYP3A4 inhibitor after an initial co-administration period of no more than fourteen days. In some embodiments, the strong CYP34A inhibitor is itraconazole.

In some embodiments, the present disclosure provides a method of treating a disease, disorder, or condition in a human in need thereof, wherein the human is being systemically administered a strong cytochrome P450 3A4 (CYP3A4) inhibitor, the method comprising: a) orally administering to the human a therapeutically effective amount of azetukalner; and b) discontinuing the systemic administration of the strong CYP3A4 inhibitor after an initial co-administration period of no more than seven days. In some embodiments, the strong CYP34A inhibitor is itraconazole.

In some embodiments, the present disclosure provides a method of treating a disease, disorder, or condition in a human in need thereof, the method comprising: a) orally administering to the human a therapeutically effective amount of azetukalner; b) subsequently determining that the human is being systemically administered a strong cytochrome P450 3A4 (CYP3A4) inhibitor; and c) discontinuing the systemic administration of the strong CYP3A4 inhibitor after an initial co-administration period of no more than fourteen days.

In some embodiments, the present disclosure provides a method of treating a disease, disorder, or condition in a human in need thereof, the method comprising: a) orally administering to the human a therapeutically effective amount of azetukalner; b) subsequently determining that the human is being systemically administered a strong cytochrome P450 3A4 (CYP3A4) inhibitor; and c) discontinuing the systemic administration of the strong CYP3A4 inhibitor after an initial co-administration period of no more than seven days.

In some embodiments, the present disclosure provides a method of treating a disease, disorder, or condition in a human in need thereof, wherein the human is being systemically administered a strong cytochrome P450 3A4 (CYP3A4) inhibitor, the method comprising: a) orally administering to the human a therapeutically effective amount of azetukalner; and b) reducing the dose of the strong CYP3A4 inhibitor being systemically administered to the human after an initial co-administration period of fourteen days with azetukalner.

In some embodiments, the present disclosure provides a method of treating a disease, disorder, or condition in a human in need thereof, wherein the human is being systemically administered a strong cytochrome P450 3A4 (CYP3A4) inhibitor, the method comprising: a) orally administering to the human a therapeutically effective amount of azetukalner; and b) reducing the dose of the strong CYP3A4 inhibitor being systemically administered to the human after an initial co-administration period of seven days with azetukalner.

In some embodiments, the present disclosure provides a method of treating a disease, disorder, or condition in a human in need thereof, the method comprising: a) orally administering to the human a therapeutically effective amount of azetukalner; b) subsequently determining that the human is being systemically administered a strong cytochrome P450 3A4 (CYP3A4) inhibitor; and c) reducing the dosage of the strong CYP3A4 inhibitor being systemically administered to the human after an initial co-administration period of fourteen days with azetukalner.

In some embodiments, the present disclosure provides a method of treating a disease, disorder, or condition in a human in need thereof, the method comprising: a) orally administering to the human a therapeutically effective amount of azetukalner; b) subsequently determining that the human is being systemically administered a strong cytochrome P450 3A4 (CYP3A4) inhibitor; and c) reducing the dosage of the strong CYP3A4 inhibitor being systemically administered to the human after an initial co-administration period of seven days with azetukalner.

In some embodiments, the present disclosure provides a method of treating a disease, disorder, or condition in a human in need thereof, wherein the human is being systemically administered a cytochrome P450 3A4 (CYP3A4) inducer, the method comprising orally administering to the human a therapeutically effective amount of azetukalner. In some embodiments, the co-administration does not require a dose adjustment of the CYP3A4 inducer. In some embodiments, the co-administration does not require discontinuation of the CYP3A4 inducer. In some embodiments, the CYP3A4 inducer is carbamazepine.

In certain embodiments, the disease, disorder, or condition is a seizure disorder, a depressive disorder, pain, or anhedonia.

In certain embodiments, the disease, disorder, or condition is a seizure disorder. In certain embodiments, the seizure disorder are seizures associated with a disease, disorder, or condition (e.g., wherein seizures/epileptic episodes are symptoms of a disease, disorder, or condition). In certain embodiments, the seizure disorder refers to focal onset epilepsy, also known as partial onset epilepsy. In some embodiments, the seizure disorder is focal onset epilepsy, photosensitive epilepsy, intractable epilepsy, benign rolandic epilepsy, CDKL5 disorder, childhood and juvenile absence epilepsy, Dravet syndrome, frontal lobe epilepsy, Glut1 deficiency syndrome, infantile spasms/West's syndrome, juvenile myoclonic epilepsy, Landau-Kleffner syndrome, Lennox-Gastaut syndrome (LGS), epilepsy with myoclonic-absences, Ohtahara syndrome, Panayiotopoulos syndrome, PCDH19 epilepsy, progressive myoclonic epilepsies, ring chromosome 20 syndrome, reflex epilepsies, temporal lobe epilepsy, Lafora progressive myoclonus epilepsy, early infantile epileptic encephalopathy, early onset epileptic encephalopathy, generalized epilepsy, generalized epilepsy with febrile seizures, paroxysmal dyskinesia, generalized onset seizures, focal onset seizures, primary generalized tonic-clonic seizures, seizures associated with self-induced syncope, seizures associated with Angelman syndrome, seizures associated with hypothalamic hamartoma, seizures associated with Rasmussen's syndrome, seizures associated with neurocutaneous syndromes, seizures associated with tuberous sclerosis complex, seizures associated with Rett syndrome, seizures associated with multiple sclerosis, seizures associated with Alzheimer's disease, seizures associated with autism, seizures associated with ataxia, or seizures associated with hypotonia, or a combination thereof. In some embodiments, the seizure disorder is focal onset epilepsy, photosensitive epilepsy, intractable epilepsy, benign rolandic epilepsy, CDKL5 disorder, childhood and juvenile absence epilepsy, Dravet syndrome, frontal lobe epilepsy, Glut1 deficiency syndrome, infantile spasms/West's syndrome, juvenile myoclonic epilepsy, Landau-Kleffner syndrome, Lennox-Gastaut syndrome (LGS), epilepsy with myoclonic-absences, Ohtahara syndrome, Panayiotopoulos syndrome, PCDH19 epilepsy, progressive myoclonic epilepsies, ring chromosome 20 syndrome, reflex epilepsies, temporal lobe epilepsy, Lafora progressive myoclonus epilepsy, early infantile epileptic encephalopathy, early onset epileptic encephalopathy, generalized epilepsy, generalized epilepsy with febrile seizures, paroxysmal dyskinesia, generalized onset seizures, focal onset seizures, or primary generalized tonic-clonic seizures, or combination thereof. In some embodiments, the seizure disorder is seizures associated with self-induced syncope, seizures associated with Angelman syndrome, seizures associated with hypothalamic hamartoma, seizures associated with Rasmussen's syndrome, seizures associated with neurocutaneous syndromes, seizures associated with tuberous sclerosis complex, seizures associated with Rett syndrome, seizures associated with multiple sclerosis, seizures associated with Alzheimer's disease, seizures associated with autism, seizures associated with ataxia, or seizures associated with hypotonia, or a combination thereof. In some embodiments, the seizure disorder refers to focal onset seizures, also known as partial onset seizures. In some embodiments, the seizure disorder is focal onset epilepsy, photosensitive epilepsy, intractable epilepsy, benign rolandic epilepsy, childhood and juvenile absence epilepsy, Dravet syndrome, frontal lobe epilepsy, infantile spasms/West's syndrome, juvenile myoclonic epilepsy, Lennox-Gastaut syndrome (LGS), epilepsy with myoclonic-absences, Ohtahara syndrome, Panayiotopoulos syndrome, PCDH19 epilepsy, progressive myoclonic epilepsies, reflex epilepsies, temporal lobe epilepsy, Lafora progressive myoclonus epilepsy, early infantile epileptic encephalopathy, early onset epileptic encephalopathy, generalized epilepsy, generalized epilepsy with febrile seizures, generalized onset seizures, focal onset seizures, or primary generalized tonic-clonic seizures, or a combination thereof. In some embodiments, the seizure disorder is photosensitive epilepsy. In some embodiments, the seizure disorder is seizures associated with self-induced syncope. In some embodiments, the seizure disorder is intractable epilepsy. In some embodiments, the seizure disorder is seizures associated with Angelman syndrome. In some embodiments, the seizure disorder is benign rolandic epilepsy. In some embodiments, the seizure disorder is CDKL5 disorder. In some embodiments, the seizure disorder is childhood and juvenile absence epilepsy. In some embodiments, the seizure disorder is Dravet syndrome. In some embodiments, the seizure disorder is frontal lobe epilepsy. In some embodiments, the seizure disorder is Glut1 deficiency syndrome. In some embodiments, the seizure disorder is seizures associated with hypothalamic hamartoma. In some embodiments, the seizure disorder is infantile spasms/West's syndrome. In some embodiments, the seizure disorder is juvenile myoclonic epilepsy. In some embodiments, the seizure disorder is Landau-Kleffner syndrome. In some embodiments, the seizure disorder is Lennox-Gastaut syndrome (LGS). In some embodiments, the seizure disorder is epilepsy with myoclonic-absences. In some embodiments, the seizure disorder is Ohtahara syndrome. In some embodiments, the seizure disorder is Panayiotopoulos syndrome. In some embodiments, the seizure disorder is PCDH19 epilepsy. In some embodiments, the seizure disorder is progressive myoclonic epilepsies. In some embodiments, the seizure disorder is seizures associated with Rasmussen's syndrome. In some embodiments, the seizure disorder is ring chromosome 20 syndrome. In some embodiments, the seizure disorder is reflex epilepsies. In some embodiments, the seizure disorder is temporal lobe epilepsy. In some embodiments, the seizure disorder is Lafora progressive myoclonus epilepsy. In some embodiments, the seizure disorder is seizures associated with neurocutaneous syndromes. In some embodiments, the seizure disorder is seizures associated with tuberous sclerosis complex. In some embodiments, the seizure disorder is early infantile epileptic encephalopathy. In some embodiments, the seizure disorder is early onset epileptic encephalopathy. In some embodiments, the seizure disorder is generalized epilepsy. In some embodiments, the seizure disorder is generalized epilepsy with febrile seizures. In some embodiments, the seizure disorder is seizures associated with Rett syndrome. In some embodiments, the seizure disorder is seizures associated with multiple sclerosis. In some embodiments, the seizure disorder is seizures associated with Alzheimer's disease. In some embodiments, the seizure disorder is seizures associated with autism. In some embodiments, the seizure disorder is seizures associated with ataxia. In some embodiments, the seizure disorder is seizures associated with hypotonia. In some embodiments, the seizure disorder is paroxysmal dyskinesia. In some embodiments, the seizure disorder is generalized onset seizures. In some embodiments, the seizure disorder is focal onset seizures (also known as partial onset seizures). In some embodiments, the generalized onset seizures are primary generalized tonic-clonic seizures. In some embodiments, the seizure disorder is primary generalized tonic-clonic seizures. In some embodiments, the seizure disorder is primary generalized tonic-clonic seizures, generalized epilepsy, or focal onset epilepsy. In some embodiments, the seizure disorder is primary generalized tonic-clonic seizures or focal onset epilepsy. In some embodiments, the seizure disorder is primary generalized tonic-clonic seizures, focal onset seizures, or focal onset epilepsy.

In certain embodiments, the disease, disorder, or condition is a depressive disorder. “Depressive disorders” are mood disorders characterized by depressed mood. In certain embodiments, the depressive disorder is major depressive disorder (MDD), disruptive mood dysregulation disorder, persistent depressive disorder, bipolar depression, postpartum depression, premenstrual dysphoric disorder (PMDD), seasonal affective disorder (SAD), atypical depression, treatment-resistant depression (TRD), depression associated with agitation or anxiety, adjustment disorder with depressed mood, or prolonged depressive reaction, or a combination thereof.

In some embodiments, the depressive disorder is major depressive disorder (MDD). In some embodiments, the depressive disorder is disruptive mood dysregulation disorder. In some embodiments, the depressive disorder is persistent depressive disorder. In some embodiments, the depressive disorder is bipolar depression. In some embodiments, the depressive disorder is postpartum depression. In some embodiments, the depressive disorder is premenstrual dysphoric disorder (PMDD). In some embodiments, the depressive disorder is seasonal affective disorder (SAD). In some embodiments, the depressive disorder is atypical depression. In some embodiments, the depressive disorder is treatment-resistant depression (TRD). In some embodiments, the depressive disorder is depression associated with agitation or anxiety. In some embodiments, the depressive disorder is adjustment disorder with depressed mood. In some embodiments, the depressive disorder is prolonged depressive reaction. In some embodiments, the depressive disorder is major depressive disorder (MDD) or bipolar depression.

In some embodiments, the depressive disorder is bipolar depression (i.e., the depressive disorder is depression associated with bipolar disorder). In some embodiments, the bipolar disorder is treatment-resistant bipolar disorder, bipolar I disorder, bipolar II disorder, cyclothymic disorder, or bipolar disorder not otherwise specified. In some embodiments, the bipolar disorder is bipolar I disorder, bipolar II disorder, or cyclothymic disorder. In certain embodiments, the depressive disorder is depression associated with bipolar I disorder and/or bipolar II disorder. In certain embodiments, the depressive disorder is depression associated with bipolar I and/or II depression.

Also contemplated by the disclosure is treatment of obsessive-compulsive disorder (OCD), panic disorder, social anxiety disorder, social phobia, agoraphobia, agoraphobia with panic disorder, hypochondriasis, post-traumatic stress disorder (PTSD), treatment-resistant bipolar disorder, generalized anxiety disorder, attention-deficit/hyperactivity disorder (ADHD), bipolar I disorder, bipolar II disorder, manic disorder, cyclothymic disorder and bipolar disorder not otherwise specified, dysthymic disorder, depressive disorder not otherwise specified, minor depression, recurrent brief depressive disorder, depressive-type psychosis, impulse-control disorders, schizophrenia, schizophreniform disorder, schizoaffective disorder, Parkinson's disease, dementia, Alzheimer's disease, Huntington's disease, Tourette's syndrome, aggression, and substance use and/or abuse, or a combination thereof.

In certain embodiments, the disease, disorder, or condition is pain. “Pain” as used herein refers to all categories of pain and includes, but is not limited to, neuropathic pain, inflammatory pain, nociceptive pain, nociplastic pain, idiopathic pain, neuralgic pain, orofacial pain, burn pain, burning mouth syndrome, somatic pain, visceral pain, myofacial pain, dental pain, cancer pain, chemotherapy pain, trauma pain, surgical pain, post-surgical pain, childbirth pain, labor pain, reflex sympathetic dystrophy, brachial plexus avulsion, neurogenic bladder, acute pain (e.g., musculoskeletal and post-operative pain), chronic pain, persistent pain, peripherally mediated pain, centrally mediated pain, chronic headache, migraine headache (e.g., with or without aura), hemiplegic migraine (e.g., familial hemiplegic migraine), conditions associated with cephalic pain, sinus headache, tension headache, phantom limb pain, peripheral nerve injury, pain following stroke, thalamic lesions, radiculopathy, HIV pain, post-herpetic pain, non-cardiac chest pain, irritable bowel syndrome, pain associated with bowel disorders and dyspepsia, osteoarthritis (OA) pa in, chronic back pain, bone pain, soft tissue pain, and combinations thereof.

In certain embodiments, the disease, disorder, or condition is anhedonia. “Anhedonia” as used herein refers to markedly diminished interest or pleasure in all, or almost all activities. Anhedonia of mild degree is sometimes referred to as hypohedonia. Social anhedonia is a type of anhedonia. “Social anhedonia” as used herein refers to a disinterest in social contact and a lack of pleasure in social situations. Social anhedonia is characterized by social withdrawal and typically manifests as an indifference to social interactions with other people. This trait is considered to be a central characteristic, as well as a predictor, of schizophrenia spectrum disorders.

In a preferred embodiment, the subject is human. In certain embodiments, the human is a poor cytochrome P450 3A4 (CYP3A4) metabolizer. Whether or not a given subject is a poor cytochrome P450 3A4 (CYP3A4) retabolizer may be readily determined by routine techniques known to those skilled in the art.

In certain embodiments, azetukalner or a pharmaceutically acceptable salt thereof (preferably, azetukalner) is orally administered. In some embodiments, azetukalner or a pharmaceutically acceptable salt thereof (preferably, azetukalner) is administered in one or more doses. In some embodiments, azetukalner or a pharmaceutically acceptable salt thereof (preferably, azetukalner) is administered once daily.

In one embodiment, the methods described herein are achieved by administering (e.g., orally) a therapeutically effective amount of azetukalner or a pharmaceutically acceptable salt thereof (preferably, azetukalner), such as from about 0.05 mg/kg to about 2.0 mg/kg. More specific representative amounts include 0.05 mg/kg, 0.10 mg/kg, 0.20 mg/kg, 0.30 mg/kg, 0.40 mg/kg, 0.5 mg/kg, 0.6 mg/kg, 0.7 mg/kg, 0.80 mg/kg, 0.90 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg and 2.0 mg/kg, or any range of amounts created by using two of the aforementioned amounts as endpoints. In some aspects, the method includes administering (e.g., orally) 0.1-1.0 mg/kg of azetukalner or a pharmaceutically acceptable salt thereof (preferably, azetukalner). In some aspects, the method includes administering (e.g., orally) 0.2-0.5 mg/kg of azetukalner or a pharmaceutically acceptable salt thereof (preferably, azetukalner).

In some embodiments, the methods described herein are achieved by administering (e.g., orally) a therapeutically effective amount of azetukalner or a pharmaceutically acceptable salt thereof (preferably, azetukalner), such as 2 to 200 mg of azetukalner or a pharmaceutically acceptable salt thereof (preferably, azetukalner) in a single or divided doses. For example, the method can include administering (e.g., orally), in a single or divided doses, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mug, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 ng, about 97 mg, about 98 mg, about 99 ng, about 100 mg, about 101 mg, about 102 mg, about 103 mg, about 104 mg, about 105 mg, about 106 mg, about 107 mg, about 108 mg, about 109 mg, about 110 mg, about 111 mg, about 112 mg, about 113 mg, about 114 mg, about 115 mg, about 116 mg, about 117 mg, about 118 mg, about 119 mg, about 120 mg, about 121 mg, about 122 mg, about 123 mg, about 124 mg, about 125 mg, about 126 mg, about 127 mg, about 129 mg, about 130 mg, about 131 mg, about 132 mg, about 133 mg, about 134 mg, about 135 mg, about 136 mg, about 137 mg, about 138 mg, about 139 mg, about 140 mg, about 141 mg, about 142 mg, about 143 mg, about 144 mg, about 145 mg, about 146 mg, about 147 mg, about 148 mg, about 149 mg, about 150 mg, about 151 mg, about 152 mg, about 153 mg, about 154 mg, about 155 mg, about 156 mg, about 157 mg, about 158 mg, about 159 mg, about 160 mg, about 161 mg, about 162 mg, about 163 mg. about 164 mg, about 165 mg, about 166 ng, about 167 mg, about 168 mg, about 169 mg, about 170 mg, about 171 mg, about 172 mg, about 173 mg, about 174 mg, about 175 mg, about 176 mg, about 177 mg, about 178 ng, about 179 mg, about 180 mg, about 181 ng, about 182 mg, about 183 mg, about 184 mg, about 185 mg, about 186 mg, about 187 mg, about 188 mg, about 189 mg, about 190 mg, about 191 mg, about 192 mg, about 193 mg, about 194 mg, about 195 mg, about 196 mg, about 197 mg, about 198 mg, about 199 mg, or about 200 mg or administering (e.g., orally) any range of amounts created by using two of the aforementioned amounts as endpoints. In some aspects, the method includes administration of 2 to 100 mg of azetukalner or a pharmaceutically acceptable salt thereof (preferably, azetukalner) in a single or divided doses. In some aspects, the method includes administration of 2 to 50 mg of azetukalner in a single or divided doses. In some aspects, method includes the administration of a single or divided dose of 10, 15, 20, 25, or 30 mg of azetukalner or a pharmaceutically acceptable salt thereof (preferably, azetukalner). In some aspects, the method includes administration of a single or divided dose of 10 mg of azetukalner or a pharmaceutically acceptable salt thereof (preferably, azetukalner). In some aspects, the method or use includes administration of a single or divided dose of 15 mg of azetukalner or a pharmaceutically acceptable salt thereof (preferably, azetukalner). In some aspects, the method includes administration of a single or divided dose of 20 mg of azetukalner or a pharmaceutically acceptable salt thereof (preferably, azetukalner). In some aspects, the method includes administration of a single or divided dose of 25 mg of azetukalner or a pharmaceutically acceptable salt thereof (preferably, azetukalner). In some aspects, the method includes administration of a single or divided dose of 30 mg of azetukalner or a pharmaceutically acceptable salt thereof (preferably, azetukalner).

In some aspects, the methods described herein, such as the method of treating a seizure disorder in a subject in need thereof, is achieved by administering (e.g., orally) at least 20 mg of azetukalner or a pharmaceutically acceptable salt thereof, such as at least 25, 30, 35, 50, 75, or 100 mg of azetukalner or a pharmaceutically acceptable salt thereof. In some embodiments, the methods described herein, such as the method of treating a seizure disorder in a subject in need thereof, is achieved by administering (e.g., orally) at least 10 mg of azetukalner or a pharmaceutically acceptable salt thereof per day, such as at least 15 mg, 20 mg, 25 ng, 30, 35, 50, 60, 75, 85, 100, 125, 150, 175, or 200 mg of azetukalner or a pharmaceutically acceptable salt thereof per day. In some embodiments, the methods described herein, such as the method of treating a seizure disorder in a subject in need thereof, is achieved by administering (e.g., orally) at least 50 mg of azetukalner or a pharmaceutically acceptable salt thereof per day, such as at least 60, 75, 85, 100, 125, 150, 175, or 200 mg of azetukalner or a pharmaceutically acceptable salt thereof per day.

In some embodiments, the methods described herein, such as the method of treating a seizure disorder in a subject in need thereof, is achieved by administering (e.g., orally) a therapeutically effective amount of azetukalner or a pharmaceutically acceptable salt thereof (preferably, azetukalner) per day, such as 5 to 1000 mg of azetukalner or a pharmaceutically acceptable salt thereof per day, such as 5 to 500 mg or 5 to 250 mg of azetukalner or a pharmaceutically acceptable salt thereof per day. For example, the method can include administering (e.g., orally) about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 ng, about 315 mg, about 320 mg, about 325 mg, about 330 mg, about 335 mg, about 340 mg, about 345 mg, about 350 mg, about 355 mg, about 360 ng, about 365 mg, about 370 mg, about 375 ng, about 380 mg, about 385 mg, about 390 mg, about 395 mg, about 400 mg, about 405 mg, about 410 mg, about 415 mg, about 420 mg, about 425 mg, about 430 mg, about 435 mg, about 440 mg, about 445 mg, about 450 mg, about 455 mg, about 460 ng, about 465 mg, about 470 mg, about 475 ng, about 480 mg, about 485 mg, about 490 mg, about 495 mg, about 500 mg. or about 1000 mg of azetukalner or a pharmaceutically acceptable salt thereof (preferably, azetukalner) per day, or administering (e.g., orally) per day a range of amounts created by using two of the aforementioned amounts as endpoints. In some aspects, the method includes orally administering 10 to 200 mg of azetukalner or a pharmaceutically acceptable salt thereof (preferably, azetukalner) per day, such as 10, 15, 20, 25, 30, 35, or 40 mg to 75, 100, 125, 150, 1-75, or 200 mg of azetukalner or a pharmaceutically acceptable salt thereof (preferably, azetukalner) per day, including 20 to 150 mg per day. In some aspects, the oral administration includes 50, 75, 100, or 125 mg of azetukalner or a pharmaceutically acceptable salt thereof (preferably, azetukalner) per day, such as 100 mg per day. In some aspects, the method includes orally administering 10 mg of azetukalner or a pharmaceutically acceptable salt thereof (preferably, azetukalner) per day. In some aspects, the method includes orally administering 15 mg of azetukalner or a pharmaceutically acceptable salt thereof (preferably, azetukalner) per day. In some aspects, the method includes orally administering 20 mg of azetukalner or a pharmaceutically acceptable salt thereof (preferably, azetukalner) per day. In some aspects, the method includes orally administering 25 mg of azetukalner or a pharmaceutically acceptable salt thereof (preferably, azetukalner) per day.

In certain instances, the above daily doses of azetukalner or a pharmaceutically acceptable salt thereof (preferably, azetukalner) are administered (e.g., orally) as multiple doses per day, such as in two, three, four, or five doses per day. For example, a daily dose of 100 mg, may be administered in five 20 mg, four 25 mg, three 33.3 mg, or two 50 mg doses throughout the day. As further examples, a daily dose of (i) 10 mug may be administered in two 5 mg doses, (ii) 15 ng may be administered in two 7.5 mg doses or three 5 mg doses, or (iii) 20 mg may be administered in two 10 mg doses or four 5 mg doses throughout the day.

In some embodiments, the above daily doses of azetukalner or a pharmaceutically acceptable salt thereof (preferably, azetukalner) or a pharmaceutically acceptable salt thereof (preferably, azetukalner) are administered (e.g., orally) as a single dose. For example, about 5, 10, 15, 20, 25, or 30 mg to about 50, 65, 75, 100, 125, or 150 mg of azetukalner or a pharmaceutically acceptable salt thereof (preferably, azetukalner) per day can be orally administered as a single dose, including 10-25 mg, 10-30 mg, and 10-40 mg per day as a single dose, such as 10-25 mg per day as a single dose. Relatedly, any of the doses of azetukalner or a pharmaceutically acceptable salt thereof (preferably, azetukalner) discussed in the preceding paragraphs may be included in a unit dosage form.

In additional embodiments, the above-discussed methods of treating a seizure disorder by administering a therapeutically effective amount of azetukalner comprises oral administration of azetukalner or a pharmaceutically acceptable salt thereof (preferably, azetukalner) to the subject under fed conditions. In some embodiments, the oral administration of azetukalner or a pharmaceutically acceptable salt thereof to a subject under fed conditions significantly enhances the bioavailability and exposure of azetukalner as compared to the oral administration of azetukalner or a pharmaceutically acceptable salt thereof to the subject under fasted conditions, as described in U.S. Pat. No. 11,135,214, which is incorporated herein by reference in its entirety. In some embodiments, the oral administration of azetukalner or a pharmaceutically acceptable salt thereof to a subject under fed conditions increases one or more pharmacokinetic parameters for azetukalner (e.g., Cₘₐₓ, AUC_{inf}, Tₘₐₓ, t½λz, etc.) as compared to when the same amount of azetukalner or a pharmaceutically acceptable salt thereof is orally administered to the subject under fasted conditions. In some embodiments, azetukalner or a pharmaceutically acceptable salt thereof (preferably, azetukalner) is administered from 30 minutes before until 2 hours after taking food. In certain embodiments, azetukalner or a pharmaceutically acceptable salt thereof (preferably, azetukalner) is administered within 15 minutes of taking food. In certain embodiments, the administration of azetukalner or a pharmaceutically acceptable salt thereof (preferably, azetukalner) to a subject is done without dose titration.

In certain embodiments, the methods described herein may administer azetukalner or a pharmaceutically acceptable salt thereof (preferably, azetukalner) in the form of a pharmaceutically acceptable oral composition that comprises azetukalner or a pharmaceutically acceptable salt thereof (preferably, azetukalner) and one or more pharmaceutically acceptable carriers or excipients. The amount of azetukalner or a pharmaceutically acceptable salt thereof (preferably, azetukalner) included in these compositions may correspond to one or more of the amounts described herein. In some embodiments, the compositions are a unit dose.

Examples of pharmaceutically acceptable oral compositions that comprise azetukalner or a pharmaceutically acceptable salt thereof (preferably, azetukalner) include solid formulations (such as tablets, capsules, lozenges, dragées, granules, powders, wafers, multi-particulates, and films), liquid formulations (such as aqueous solutions, elixirs, tinctures, slurries, suspensions, and dispersions), and aerosolized formulations (such as mists and sprays). In one embodiment, a pharmaceutically acceptable oral composition of azetukalner or a pharmaceutically acceptable salt thereof (preferably, azetukalner) includes a pediatric suspension or granulate. All above-noted amounts of azetukalner or a pharmaceutically acceptable salt thereof (preferably, azetukalner) may be included in such formulations, e.g., a capsule comprising 5, 10, 15, 10, 25, 30, or 35 mg of azetukalner or a pharmaceutically acceptable salt thereof. In some embodiments, azetukalner or a pharmaceutically acceptable salt thereof (preferably, azetukalner) is administered in the form of a capsule. In some embodiments, 10 mg of azetukalner or a pharmaceutically acceptable salt thereof (preferably, azetukalner) is administered in the form of a capsule. In some embodiments, 15 mg of azetukalner or a pharmaceutically acceptable salt thereof (preferably, azetukalner) is administered in the form of a capsule. In some embodiments, 20 mg of azetukalner or a pharmaceutically acceptable salt thereof (preferably, azetukalner) is administered in the form of a capsule. In some embodiments, 25 mg of azetukalner or a pharmaceutically acceptable salt thereof (preferably, azetukalner) is administered in the form of a capsule.

Additional embodiments and examples of the present disclosure are described herein. These embodiments and examples are illustrative and should not be construed as limiting the scope of the claimed invention.

### EXAMPLES

In order that the present disclosure may be more fully understood, the following examples are set forth. The synthetic and biological examples described in this application are offered to illustrate the compounds, pharmaceutical compositions, and methods provided herein and are not to be construed in any way as limiting in their scope.

A Phase 1, single center, open-label, 2-part, 2-period, fixed sequence, drug-drug interaction study was conducted to evaluate the effect of multiple doses of ITZ (itraconazole; a strong CYP3A4 inhibitor) or CBZ (carbamazepine; a strong CYP3 A4 inducer) on the pharmacokinetic (PK) profile of azetukalner in healthy subjects. The study population consisted of sixty subjects, either male or female, between the ages of 18 to 55 years of age (inclusive). Subjects were separated into two cohorts of 30 subjects each to participate in either Part 1 (ITZ coadministration studies with azetukalner) or Part 2 (CBZ coadministration studies with azetukalner). Azetukalner was administered as a single oral dosing of a 25 mg capsule. Part 1 and Part 2 each consisted of two treatment periods (i.e., period 1 and period 2). Accordingly, subjects returned to the clinical research unit (CRU) for 2 treatment periods, separated by 28 days between the Period 1 dose of azetukalner and the first dose of Period 2 of Part 1 (ITZ) or Part 2 (CBZ). Blood samples for PK assessments were collected at scheduled time points during the study.

### Part 1: ITZ

### Period 1

Azetukalner was administered on Study Day 1. Subjects were confined to the CRU until Study Day 3 and were discharged subsequent to collection of the 48-hour postdose PK sample and the completion of the scheduled study assessments. Outpatient clinic visits for PK blood sample collections occurred on Study Day 6: 120 hours post-azetukalner dose, Study Day 11: 240 hours post-azetukalner dose, Study Day 21: 480 hours post-azetukalner dose. The PK sample for the 672-hour postdose time point for Period 1 coincided with the Period 2 Study Day 1 pre-ITZ dose blood collection.

### Period 2:

Subjects returned to the clinic for Period 2 of Part 1 on Study Day −1. Subject dosing proceeded from Study Day 1 through Study Day 14, whereby ITZ was administered once daily (2×100 mg oral capsule), and ITZ and azetukalner were coadministered on Study Day 4. Dosing regimen: Study Day 1, ITZ 400 mg (loading dose); Study Day 2-3, ITZ 200 mg (once daily); Study Day 4, ITZ 200 mg (once daily) coadministered with 25 mg azetukalner (single dose); and Study Day 5-14, ITZ 200 mg (once daily). Subjects were discharged on Study Day 14. Subjects returned to the clinic on the following days for outpatient collection of PK blood samples: Study Day 24: 480 hours post-azetukalner dose, and Study Day 32: 672 hours post-azetukalner dose. A follow-up call was made to the subjects on Study Day 42 (±3 days).

### Part 2: CBZ

### Period 1

Azetukalner was administered on Study Day 1. Subjects were confined to the CRU until Study Day 3 and were discharged subsequent to collection of the 48-hour postdose PK sample and the completion of the scheduled study assessments. Outpatient clinic visits for PK blood sample collections occurred on Study Day 6: 120 hours post-azetukalner dose, Study Day 11: 240 hours post-azetukalner dose, Study Day 21: 480 hours post-azetukalner dose. The PK sample for the 672-hour postdose time point for Period 1 coincided with the Period 2 Study Day 1 pre-CBZ dose blood collection.

### Period 2:

Subjects returned to the clinic for Period 2 on Study Day −1. Subject dosing proceeded from Study Day 1 through Study Day 21, whereby CBZ was administered twice daily (morning and evening), and CBZ and azetukalner were coadministered on Study Day 14. Dosing regimen: Study Day 1-3, CBZ 100 mg (twice daily): Study Day 4-6, CBZ 200 mg (twice daily); Study Day 7-13, CBZ 300 mg (twice daily); Study Day 14, CBZ 300 mg (twice daily) coadministered with 25 mg azetukalner (single dose); and Study Day 15-21, CBZ 300 mg (twice daily). Subjects were discharged on Study Day 22, approximately 12 hours after having been administered their last dose of CBZ, and upon completion of scheduled study assessments. Subjects returned to the clinic on the following days for outpatient collection of PK blood samples: Study Day 24: 240 hours post-azetukalner dose; Study Day 34: 480 hours post-azetukalner dose; and Study Day 42: 672 hours post-azetukalner dose. A follow-up call was made to the subjects on Study Day 49 (±3 days).

Data from this study showed that coadministration with the strong CYP3A4 inhibitor itraconazole increased single-dose azetakalner Cₘₐₓ by approximately 13%, and AUC_{0-∞}. by approximately 2-fold, while coadministration with the strong CYP3A4 inducer carbamazepine decreased single-dose azetukalner Cₘₐₓ by approximately 18%, and AUC. by approximately 46%. Physiologically based pharmacokinetic (PBPK) modeling of azetukalner at steady-state with prolonged systemic treatment with a strong CYP3A4 inhibitor predicted up to 5-fold increases in azetukalner mean Cₘₐₓ following the systemic treatment of the strong CYP3A4 inhibitor for ˜3 months, while systemic treatment for 1 week predicted a transient 1.6-fold increase in azetukalner mean Cₘₐₓ. Further Population pharmacokinetic (popPK) modeling of azetukalner predicted that coadministration with two or more CYP3A4 inducers can reduce the Cₘₐₓ and the AUC₀₋₂₄ of azetukalner by approximately 27%, and approximately 38%, respectively. Thus, it was determined that concomitant systemic treatment with either a strong CYP3A4 inhibitor or inducer is permissible for a given co-administration period.

### INCORPORATION BY REFERENCE

The present application refers to various issued patent, published patent applications, scientific journal articles, and other publications, all of which are incorporated herein by reference. The details of one or more embodiments of the present disclosure are set forth herein. Other features, objects, and advantages of the present disclosure will be apparent from the Detailed Description, the FIGS., the Examples, and the Claims

### EQUIVALENTS AND SCOPE

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure includes embodiments in which more than one, or all of the group members are present in, employed in, or otherwise relevant to a given product or process.

Furthermore, the present disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the present disclosure, or aspects of the present disclosure, is/are referred to as comprising particular elements and/or features, certain embodiments of the present disclosure or aspects of the present disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the present disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the present disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

1. A method of treating a disease, disorder, or condition in a human in need thereof, wherein the human is being administered a cytochrome P450 3A4 (CYP3A4) inhibitor, the method comprising:
a) administering to the human a therapeutically effective amount of azetukalner; and
b) discontinuing the administration of the CYP3A4 inhibitor.

2. A method of treating a disease, disorder, or condition in a human in need thereof, wherein the human is being administered a cytochrome P450 3A4 (CYP3A4) inhibitor, the method comprising:
a) discontinuing the administration of the CYP3A4 inhibitor; and
b) administering to the human a therapeutically effective amount of azetukalner, thereby avoiding the use of the CYP3A4 inhibitor in combination with azetukalner.

3. A method of treating a disease, disorder, or condition in a human in need thereof, the method comprising:
a) administering to the human a therapeutically effective amount of azetukalner;
b) subsequently determining that the human is being administered a cytochrome P450 3A4 (CYP3A4) inhibitor; and
c) discontinuing the administration of the CYP3A4 inhibitor.

4-31. (canceled)

32. The method of claim 1, comprising discontinuing administration of the CYP3A4 inhibitor after an initial co-administration/concomitant treatment period with azetukalner.

33. The method of claim 32, wherein the initial co-administration period is no more than fourteen days.

34. The method of claim 32, wherein the initial co-administration period is no more than ten days.

35. The method of claim 32, wherein the initial co-administration period is no more than nine days.

36. The method of claim 32, wherein the initial co-administration period is no more than eight days.

37. The method of claim 32, wherein the initial co-administration period is no more than seven days.

38-43. (canceled)

44. The method of claim 32, wherein the initial co-administration period is between one and fourteen days.

45-51. (canceled)

52. The method of claim 32, wherein the initial co-administration period is fourteen days.

53. The method of claim 32, wherein the initial co-administration period is seven days.

54. (canceled)

55. The method of claim 1, wherein the CYP3A4 inhibitor is systemically administered.

56-64. (canceled)

65. The method of claim 1, wherein the azetukalner is orally administered to the human.

66-71. (canceled)

72. The method of claim 1, wherein the disease, disorder, or condition is a seizure disorder, a depressive disorder, pain, or anhedonia.

73. The method of claim 1, wherein the disease, disorder, or condition is a seizure disorder selected from the group consisting of partial onset (focal) seizures, photosensitive epilepsy, seizures associated with self-induced syncope, intractable epilepsy, seizures associated with Angelman syndrome, benign rolandic epilepsy, CDKL5 disorder, childhood and juvenile absence epilepsy, Dravet syndrome, frontal lobe epilepsy, Glut1 deficiency syndrome, seizures associated with hypothalamic hamartoma, infantile spasms/West's syndrome, juvenile myoclonic epilepsy, Landau-Kleffner syndrome, Lennox-Gastaut syndrome (LGS), epilepsy with myoclonic-absences, Ohtahara syndrome, Panayiotopoulos syndrome, PCDH19 epilepsy, progressive myoclonic epilepsies, seizures associated with Rasmussen's syndrome, ring chromosome 20 syndrome, reflex epilepsies, temporal lobe epilepsy, Lafora progressive myoclonus epilepsy, seizures associated with neurocutaneous syndromes, seizures associated with tuberous sclerosis complex, early infantile epileptic encephalopathy, early onset epileptic encephalopathy, generalized epilepsy with febrile seizures+, seizures associated with Rett syndrome, seizures associated with multiple sclerosis, seizures associated with Alzheimer's disease, seizures associated with autism, seizures associated with ataxia, seizures associated with hypotonia, paroxysmal dyskinesia, generalized onset seizures, focal onset seizures, and primary generalized tonic-clonic seizures.

74. The method of claim 1, wherein the disease, disorder, or condition is focal onset epilepsy.

75-79. (canceled)

80. The method of claim 1, where the dosage of azetukalner orally administered to the human is 10 mg, 15 mg, 20 mg, 25 mg, or 30 mg.

81-83. (canceled)

84. The method of claim 1, where the dosage of azetukalner orally administered to the human is 10 mg per day, 15 mg per day, 20 mg per day, 25 mg per day, or 30 mg per day.

85. The method of claim 1, where the dosage of azetukalner orally administered to the human is 10 to 30 mg per day as a single dose.

86. (canceled)

87. The method of claim 1, where the azetukalner is administered to the human once per day.

88. (canceled)
